(19) United States
(12) Patent Application Publication
(10) Pub. No.: US 2003/0211515 A1
(43) Pub. Date: Nov. 13, 2003

(54) Novel compounds

(76) Inventors: Amy K Lasek, Oakland, CA (US); Henry Yue, Sunnyvale, CA (US); Chandra S Arvizu, San Jose, CA (US)

Correspondence Address: Incyte Genomics Inc, Legal Department, 3160 Porter Drive, Palo Alto, CA, 94304, US

(21) Appl. No.: 10/312,162

(22) PCT Filed: Jun. 29, 2001
(86) PCT No.: PCT/US01/20729

(51) Int. Cl. C07H 21/04; C12Q 1/68; C07K 14/47; C12P 21/02; C12N 5/06

(52) U.S. Cl. 435/6; 435/69.1; 435/320.1; 435/325; 530/350; 536/23.5

(57) ABSTRACT

Polypeptides and polynucleotides of the genes set forth in Table 1 and methods for producing such polypeptides by recombinant techniques are disclosed. Also disclosed are methods for utilizing polypeptides and polynucleotides of the genes set forth in Table 1 in diagnostic assays.

This application claims the benefit of provisional application U.S. Serial No. 60/215,454 filed Jun. 30, 2000, all of which application is hereby incorporated by reference herein.

### TECHNICAL FIELD

This invention relates to a cDNA which encodes an extracellular matrix (ECM)-related cancer marker and to the use of the cDNA and the encoded protein in the diagnosis and treatment of cancers, in particular, colon and lung cancer.

### BACKGROUND OF THE INVENTION

Phylogenetic relationships among organisms have been demonstrated many times, and studies from a diversity of prokaryotic and eukaryotic organisms suggest a more or less gradual evolution of molecules, biochemical and physiological mechanisms, and metabolic pathways. Despite different evolutionary pressures, the proteins of nematode, fly, rat, and man have common chemical and structural features and generally perform the same cellular function. Comparisons of the nucleic acid and protein sequences from organisms where structure and/or function are known accelerate the investigation of human sequences and allow the development of model systems for testing diagnostic and therapeutic agents for human conditions, diseases, and disorders.

Cancers and malignant tumors are characterized by continuous cell proliferation and cell death and are causally related to both genetics and the environment. Cancer markers are of great importance in determining familial predisposition to cancers and in the early diagnosis and prognosis of various cancers.

Colorectal cancer is the fourth most common cancer and the second most common cause of cancer death in the United States with approximately 130,000 new cases and 55,000 deaths per year. Colon and rectal cancers share many environmental risk factors and both are found in individuals with specific genetic syndromes. (See Potter, JD (1999) J Natl Cancer Institute 91:916-932 for a review of colorectal cancer.) Colon cancer is the only cancer that occurs with approximately equal frequency in men and women, and the five-year survival rate following diagnosis of colon cancer is around 55% in the United States (Ries et al. (1990) National Institutes of Health, DHHS Publ No. (NIH)90-2789).

Colon cancer is causally related to both genes and the environment. Several molecular pathways have been linked to the development of colon cancer, and the expression of key genes in any of these pathways may be lost by inherited or acquired mutation or by hypermethylation. There is a particular need to identify genes for which changes in expression may provide an early indicator of colon cancer or a predisposition for the development of colon cancer, as well as potential therapeutic targets for treatment of the disease.

A number of genes associated with the predisposition, development, and progression of colon cancer have been identified. For example, it is well known that abnormal patterns of DNA methylation occur consistently in human tumors. In colon cancer in particular, it has been found that these changes occur early in tumor progression such as in premalignant polyps that precede colon cancer. DNA methyltransferase, the enzyme that performs DNA methylation, is significantly increased in histologically normal mucosa from patients with colon cancer or the benign polyps that precede cancer, and this increase continues during the progression of colonic neoplasms (Wafik et al. (1991) Proc Natl Acad Sci USA 88:3470-3474). Familial Adenomatous Polyposis (FAP) is a rare autosomal dominant syndrome that precedes colon cancer and is caused by an inherited mutation in the adenomatous polyposis coli (APC) gene. The APC gene is a part of the APC-β-catenin-Tcf (T-cell factor) pathway. Impairment of this pathway results in the loss of orderly replication, adhesion, and migration of colonic epithelial cells that results in the growth of polyps. Hereditary nonpolyposis Colorectal Cancer (HNPCC) is another inherited autosomal dominant syndrome that is distinguished by the tendency to early onset of colon cancer and the development of other cancers. HNPCC results from the mutation of one or more genes in the DNA mis-match repair (MMR) pathway. Mutations in two human MMR genes, MSH2 and MLH1, are found in a large majority of HNPCC families identified to date. Almost all colon cancers arise from cells in which the estrogen receptor (ER) gene has been silenced. The silencing of ER gene transcription is age related and linked to hypermethylation of the ER gene (Issa et al. (1994) Nature Genetics 7:536-540). Introduction of an exogenous ER gene into cultured colon carcinoma cells results in marked growth suppression.

Clearly there are a number of genetic alterations associated with colon cancer and with the development and progression of the disease that potentially provide early indicators of cancer development, and which may also be used to monitor disease progression or provide therapeutic targets.

The extracellular matrix (ECM) is a complex network of glycoproteins, polysaccharides, proteoglycans, and other macromolecules that are secreted from a cell, and provides cells with a mechanical scaffold for adhesion, migration and signal transduction. Tumor cells often overcome the need for cell-ECM anchorage and ECM-mediated growth control in the process of becoming metastatic (Ruoslahti, E. (1996) Sci. Am. 275:72-77.) Thus alterations in ECM proteins provides an additional source of markers and potential therapeutic targets for some forms of cancer. Many ECM proteins are characterized by the presence of transmembrane domains and protein-protein or cell-matrix interaction domains such as leucine-rich repeats (LLR) believed to be associated with signal transduction and cellular adhesion as well as in protein-protein interactions (Gay, N. J., et al. (1991) FEBS Lett. 29:87-91).

The discovery of a cDNA encoding ECM-related protein satisfies a need in the art by providing compositions which are useful in the diagnosis and treatment of cancer particularly colon and lung cancer.

### SUMMARY OF THE INVENTION

The invention is based on the discovery of a cDNA encoding ECM-related protein, ECMRP, which is useful in the diagnosis and treatment of cancer, particularly colon and lung cancer.

The invention provides an isolated cDNA comprising a nucleic acid sequence encoding a protein having the amino acid sequence of SEQ ID NO:1. The invention also provides an isolated CDNA or the complement thereof selected from the group consisting of a nucleic acid sequence of SEQ ID NO:2, a fragment of SEQ ID NO:2 selected from SEQ ID NOs:3-13, and a variant of SEQ ID NO:2 selected from SEQ ID NOs:14-15. The invention additionally provides a composition, a substrate, and a probe comprising the cDNA, or the complement of the cDNA, encoding ECM-related protein. The invention further provides a vector containing the cDNA, a host cell containing the vector and a method for using the cDNA to make ECM-related protein. The invention still further provides a transgenic cell line or organism comprising the vector containing the cDNA encoding ECM-related protein. The invention additionally provides a fragment, a variant, or the complement of the cDNA selected from the group consisting of SEQ ID NOs:2-15. In one aspect, the invention provides a substrate containing at least one of these fragments or variants or the complements thereof. In a second aspect, the invention provides a probe comprising a cDNA or the complement thereof which can be used in methods of detection, screening, and purification. In a further aspect, the probe is a single-stranded complementary RNA or DNA molecule.

The invention provides a method for using a cDNA to detect the differential expression of a nucleic acid in a sample comprising hybridizing a probe to the nucleic acids, thereby forming hybridization complexes and comparing hybridization complex formation with a standard, wherein the comparison indicates the differential expression of the cDNA in the sample. In one aspect, the method of detection further comprises amplifying the nucleic acids of the sample prior to hybridization. In another aspect, the method showing differential expression of the cDNA is used to diagnose cancer, in particular, colon and lung cancer. In another aspect, the cDNA or a fragment or a variant or the complements thereof may comprise an element on an array.

The invention additionally provides a method for using a CDNA or a fragment or a variant or the complements thereof to screen a library or plurality of molecules or compounds to identify at least one ligand which specifically binds the cDNA, the method comprising combining the cDNA with the molecules or compounds under conditions allowing specific binding, and detecting specific binding to the cDNA, thereby identifying a ligand which specifically binds the cDNA. In one aspect, the molecules or compounds are selected from DNA molecules, RNA molecules, peptide nucleic acids, artificial chromosome constructions, peptides, transcription factors, repressors, and regulatory molecules.

The invention provides a purified protein or a portion thereof selected from the group consisting of an amino acid sequence of SEQ ID NO:1, a variant having at least 85% identity to the amino acid sequence of SEQ ID NO:1, an antigenic epitope of SEQ ID NO:1, and a biologically active portion of SEQ ID NO:1. The invention also provides a composition comprising the purified protein and a pharmaceutical carrier. The invention still further provides a method for using a protein to screen a library or a plurality of molecules or compounds to identify at least one ligand, the method comprising combining the protein with the molecules or compounds under conditions to allow specific binding and detecting specific binding, thereby identifying a ligand which specifically binds the protein. In one aspect, the molecules or compounds are selected from DNA molecules, RNA molecules, peptide nucleic acids, peptides, proteins, mimetics, agonists, antagonists, antibodies, immunoglobulins, inhibitors, and drugs. In another aspect, the ligand is used to treat a subject with cancer, in particular, colon and lung cancer.

The invention provides a method of using a protein to screen a subject sample for antibodies which specifically bind the protein comprising isolating antibodies from the subject sample, contacting the isolated antibodies with the protein under conditions that allow specific binding, dissociating the antibody from the bound-protein, and comparing the quantity of antibody with known standards, wherein the presence or quantity of antibody is diagnostic of a cancer, in particular, colon and lung cancer.

The invention also provides a method of using a protein to prepare and purify antibodies comprising immunizing a animal with the protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified antibodies.

The invention provides a purified antibody which binds specifically to a protein which is expressed in a cancer, in particular, colon and lung cancer. The invention also provides a method of using an antibody to diagnose a cancer, in particular, colon and lung cancer comprising combining the antibody comparing the quantity of bound antibody to known standards, thereby establishing the presence of a cancer, in particular, colon and lung cancer. The invention further provides a method of using an antibody to treat a cancer, in particular, colon and lung cancer comprising administering to a patient in need of such treatment a composition comprising the purified antibody and a pharmaceutical carrier.

The invention provides a method for inserting a heterologous marker gene into the genomic DNA of a mammal to disrupt the expression of the endogenous polynucleotide. The invention also provides a method for using a cDNA to produce a mammalian model system, the method comprising constructing a vector containing the cDNA selected from SEQ ID NOs:2-15, transforming the vector into an embryonic stem cell, selecting a transformed embryonic stem cell, microinjecting the transformed embryonic stem cell into a mammalian blastocyst, thereby forming a chimeric blastocyst, transferring the chimeric blastocyst into a pseudopregnant dam, wherein the dam gives birth to a chimeric offspring containing the CDNA in its germ line, and breeding the chimeric mammal to produce a homozygous, mammalian model system.

### BRIEF DESCRIPTION OF THE FIGURES AND TABLE

FIGS. 1A through 1K show the ECM-related protein (SEQ ID NO:1) encoded by the cDNA (SEQ ID NO:2). The alignment was produced using MACDNASIS PRO software (Hitachi Software Engineering, South San Francisco Calif.).

FIG. 2 shows the expression of ECMRP in various normal adult tissues. The X-axis indicates the tissue type, and the Y-axis the expression of ECMRP relative to that found in normal colon tissue (e.g., 100%). The analysis was performed by TAQMAN (Applied Biosystems, Foster City Calif.).

FIG. 3 shows the differential expression of ECMRP in tissues from patients with colon cancer relative to normal colon tissue. The X-axis indicates the patient ID (Donor ID), and the Y-axis the expression ECMRP relative to that observed in tumor tissue from Donor ID 3582 (e.g., 100%). Tumor samples are displayed in black, and normal tissue in white. The analysis was performed by TAQMAN (Applied Biosystems).

Table 1 shows the Northern analysis for ECMRP produced using the LIFESEQ Gold database (Incyte Genomics, Palo Alto Calif.). The first column presents the tissue categories; the second column, the number of clones in the tissue category; the third column, the number of libraries in which at least one transcript was found relative to the total number of libraries in that category; the fourth column, the absolute abundance of the transcript (number of transcripts); and the fifth column, percent abundance of the transcript.

Table 2 shows the differential expression of ECMRP in tissues from patients with colon and lung cancer relative to normal colon or lung tissue as determined by array analysis. The first column lists the differential expression (DE) between the tumor sample and normal tissue. The results are expressed in terms of the ratio of tumor/normal expression. Column 2 (P1 Description) lists the tissue and patient donor (Dn) for microscopically normal samples labeled with fluorescent green dye Cy3. Column 3 (P2 Description) lists the tissue and patient donor (Dn) for diseased samples (colon tumor or colon polyps, lung tumor) labeled with fluorescent red dye Cy5.

### DESCRIPTION OF THE INVENTION

It is understood that this invention is not limited to the particular machines, materials and methods described. It is also to be understood that the terminology used herein is for the purpose of describing particular embodiments and is not intended to limit the scope of the present invention which will be limited only by the appended claims. As used herein, the singular forms “a”, “an”, and “the” include plural reference unless the context clearly dictates otherwise. For example, a reference to “a host cell” includes a plurality of such host cells known to those skilled in the art.

Unless defined otherwise, all technical and scientific terms used herein have the same meanings as commonly understood by one of ordinary skill in the art to which this invention belongs. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“ECM-related protein” refers to a purified protein obtained from any mammalian species, including bovine, canine, murine, ovine, porcine, rodent, simian, and preferably the human species, and from any source, whether natural, synthetic, semi-synthetic, or recombinant.

“Array” refers to an ordered arrangement of at least two cDNAs or antibodies on a substrate. At least one of the cDNAs or antibodies represents a control or standard, and the other, a cDNA or antibody of diagnostic or therapeutic interest. The arrangement of two to about 40,000 cDNAs or of two to about 40,000 monoclonal or polyclonal antibodies on the substrate assures that the size and signal intensity of each labeled hybridization complex, formed between each cDNA and at least one nucleic acid, or antibody:protein complex, formed between each antibody and at least one protein to which the antibody specifically binds, is individually distinguishable.

The “complement” of a cDNA of the Sequence Listing refers to a nucleic acid molecule which is completely complementary over its full length and which will hybridize to the cDNA or an MRNA under conditions of high stringency.

“cDNA” refers to an isolated polynucleotide, nucleic acid molecule, or any fragment or complement thereof. It may have originated recombinantly or synthetically, may be double-stranded or single-stranded, represents coding and noncoding 3′ or 5′ sequence, and lacks introns.

The phrase “cDNA encoding a protein” refers to a nucleotide sequence that closely aligns with sequences which encode conserved regions, motifs or domains that were identified by employing analyses well known in the art. These analyses include BLAST (Basic Local Alignment Search Tool) which provides identity within the conserved region (Altschul (1993) J Mol Evol 36: 290-300; Altschul et al. (1990) J Mol Biol 215:403-410).

A “composition” refers to the polynucleotide and a labeling moiety , a purified protein and a pharmaceutical carrier, an antibody and a labeling moiety, and the like.

“Derivative” refers to a cDNA or a protein that has been subjected to a chemical modification. Derivatization of a cDNA can involve substitution of a nontraditional base such as queosine or of an analog such as hypoxanthine. These substitutions are well known in the art. Derivatization of a protein involves the replacement of a hydrogen by an acetyl, acyl, alkyl, amino, formyl, or morpholino group. Derivative molecules retain the biological activities of the naturally occurring molecules but may confer advantages such as longer lifespan or enhanced activity.

“Differential expression” refers to an increased or upregulated or a decreased or downregulated expression as detected by absence, presence, or at least two-fold change in the amount of transcribed messenger RNA or translated protein in a sample.

“Disorder” refers to conditions, diseases or syndromes in which the cDNAs and ECM-related protein are differentially expressed. Such a disorder includes cancer, and in particular, colon and lung cancer.

“Fragment” refers to a chain of consecutive nucleotides from about 50 to about 4000 base pairs in length. Fragments may be used in PCR or hybridization technologies to identify related nucleic acid molecules and in binding assays to screen for a ligand. Such ligands are useful as therapeutics to regulate replication, transcription or translation.

A “hybridization complex” is formed between a cDNA and a nucleic acid of a sample when the purines of one molecule hydrogen bond with the pyrimidines of the complementary molecule, e.g., 5′-A-G-T-C-3′ base pairs with 3′-T-C-A-G-5′. Hybridization conditions, degree of complementarity and the use of nucleotide analogs affect the efficiency and stringency of hybridization reactions.

“Labeling moiety” refers to any visible or radioactive label than can be attached to or incorporated into a cDNA or protein. Visible labels include but are not limited to anthocyanins, green fluorescent protein (GFP), S glucuronidase, luciferase, Cy3 and Cy5, and the like. Radioactive markers include radioactive forms of hydrogen, iodine, phosphorous, sulfur, and the like.

“Ligand” refers to any agent, molecule, or compound which will bind specifically to a polynucleotide or to an epitope of a protein. Such ligands stabilize or modulate the activity of polynucleotides or proteins and may be composed of inorganic and/or organic substances including minerals, cofactors, nucleic acids, proteins, carbohydrates, fats, and lipids.

“Oligonucleotide” refers a single-stranded molecule from about 18 to about 60 nucleotides in length which may be used in hybridization or amplification technologies or in regulation of replication, transcription or translation. Equivalent terms are amplimer, primer, and oligomer.

An “oligopeptide” is an amino acid sequence from about five residues to about 15 residues that is used as part of a fusion protein to produce an antibody.

“Portion” refers to any part of a protein used for any purpose; but especially, to an epitope for the screening of ligands or for the production of antibodies.

“Post-translational modification” of a protein can involve lipidation, glycosylation, phosphorylation, acetylation, racemization, proteolytic cleavage, and the like These processes may occur synthetically or biochemically. Biochemical modifications will vary by cellular location, cell type, pH, enzymatic milieu, and the like.

“Probe” refers to a cDNA that hybridizes to at least one nucleic acid in a sample. Where targets are single-stranded, probes are complementary single strands. Probes can be labeled with reporter molecules for use in hybridization reactions including Southern, northern, in situ, dot blot, array, and like technologies or in screening assays.

“Protein” refers to a polypeptide or any portion thereof. A “portion” of a protein refers to that length of amino acid sequence which would retain at least one biological activity, a domain identified by PFAM or PRINTS analysis or an antigenic epitope of the protein identified using Kyte-Doolittle algorithms of the PROTEAN program (DNASTAR, Madison Wis.).

“Purified” refers to any molecule or compound that is separated from its natural environment and is from about 60% free to about 90% free from other components with which it is naturally associated.

“Sample” is used in its broadest sense as containing nucleic acids, proteins, antibodies, and the like. A sample may comprise a bodily fluid; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue; a tissue print; a fingerprint, buccal cells, skin, or hair; and the like.

“Similarity” as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197) or BLAST2 (Altschul et al. (1997) Nucleic Acids Res 25:3389-3402). BLAST2 may be used in a standardized and reproducible way to insert gaps in one of the sequences in order to optimize alignment and to achieve a more meaningful comparison between them. Particularly in proteins, similarity is greater than identity in that conservative substitutions, for example, valine for leucine or isoleucine, are counted in calculating the reported percentage. Substitutions which are considered to be conservative are well known in the art.

“Specific binding” refers to a special and precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which cDNAs or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

“Variant” refers to molecules that are recognized variations of a cDNA or a protein encoded by the cDNA. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

### THE INVENTION

The invention is based on the discovery of a cDNA which encodes ECM-related protein and on the use of the cDNA, or fragments thereof, and protein, or portions thereof, directly or as compositions in the characterization, diagnosis, and treatment of colon and lung cancer.

Nucleic acids encoding the ECM-related protein of the present invention were first identified in Incyte Clone 2743093 using a computer search for nucleotide and/or amino acid sequence alignments. SEQ ID NO:2 was derived from the following overlapping and/or extended nucleic acid sequences (SEQ ID NO:3-13): Incyte Clones 2743093H1 (SKINDIA01), 4876623F9 (COLDNOT01), 2316239T6 (OVARNOT02), 6258015F8 (BMARTXT06), and shotgun sequences 7677606J1, 71111915V1, 71112850V1, 71262960V1, 71264035V1, 71113484V1, and 71114738V1.

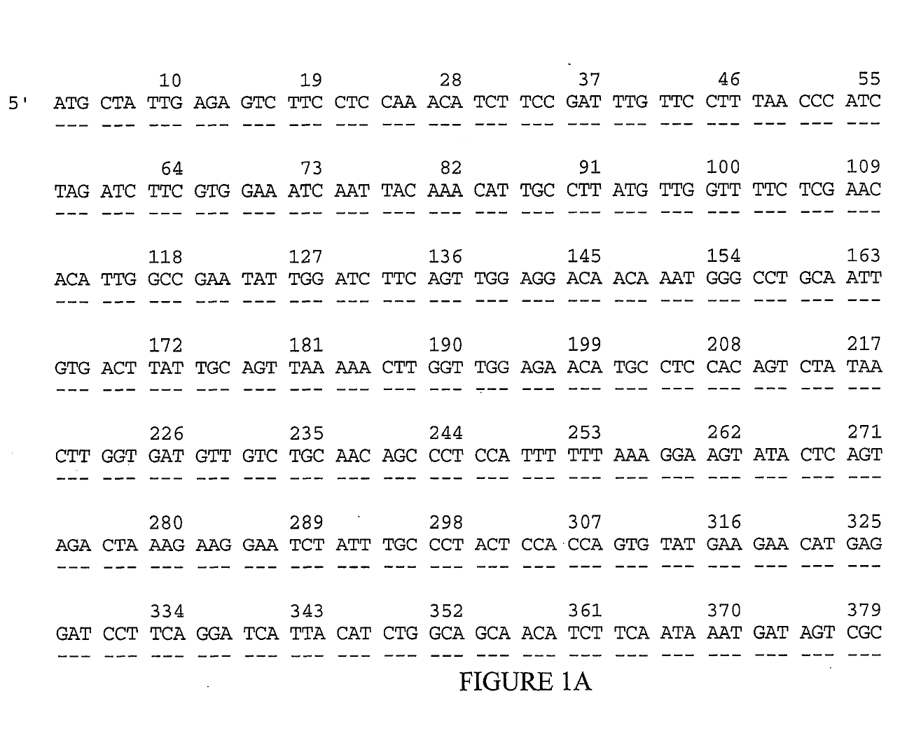

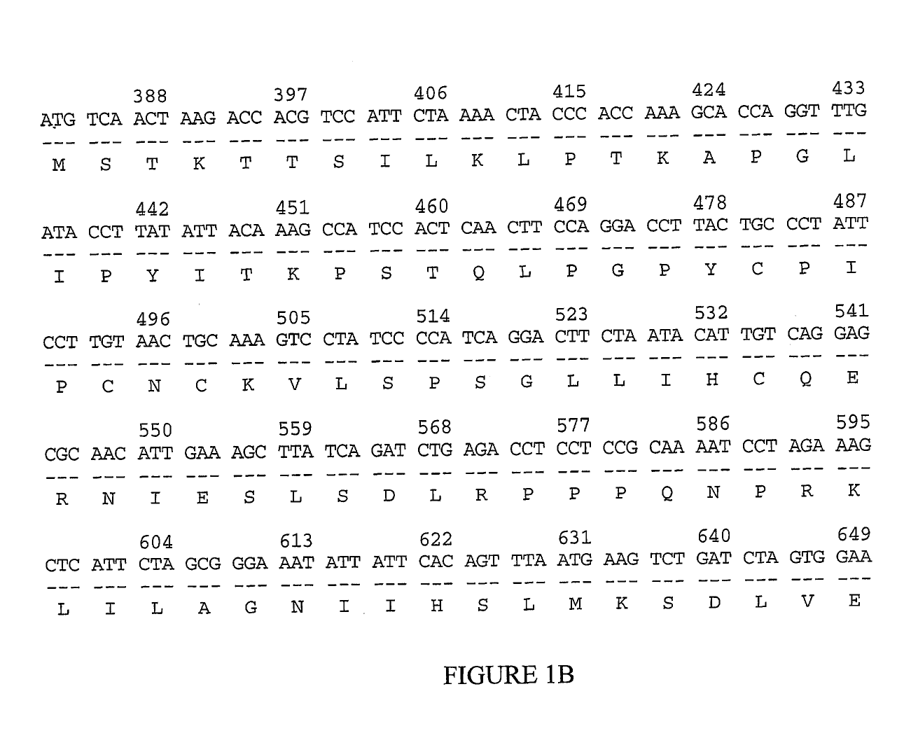

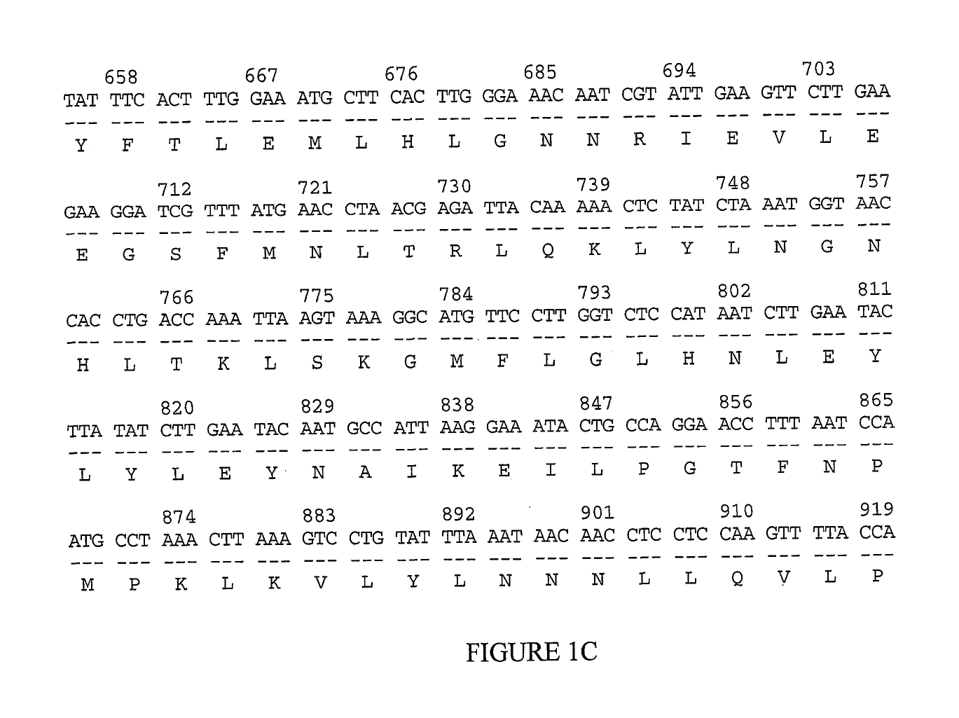

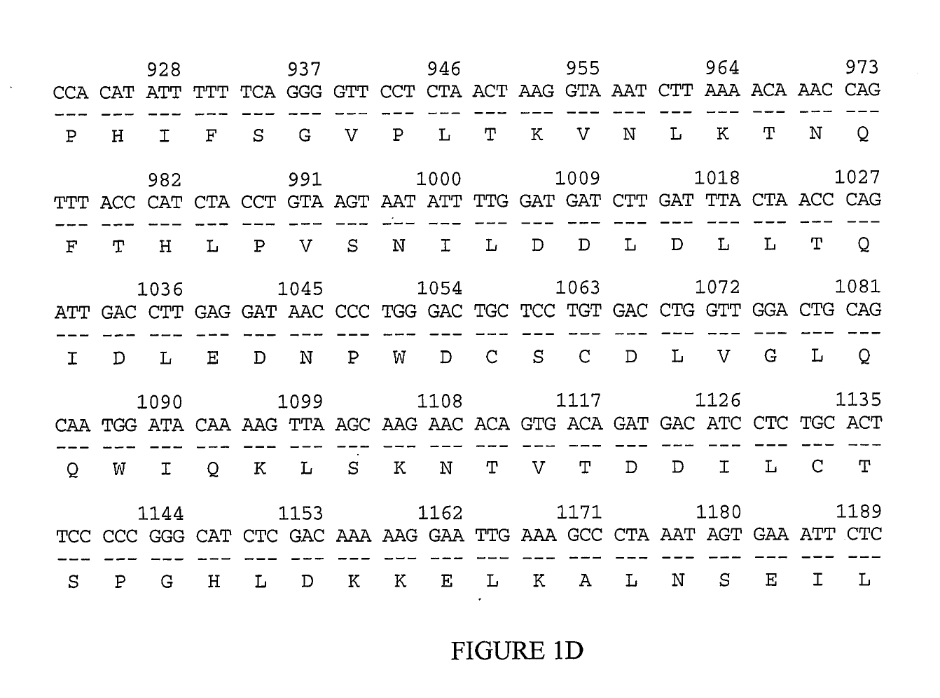

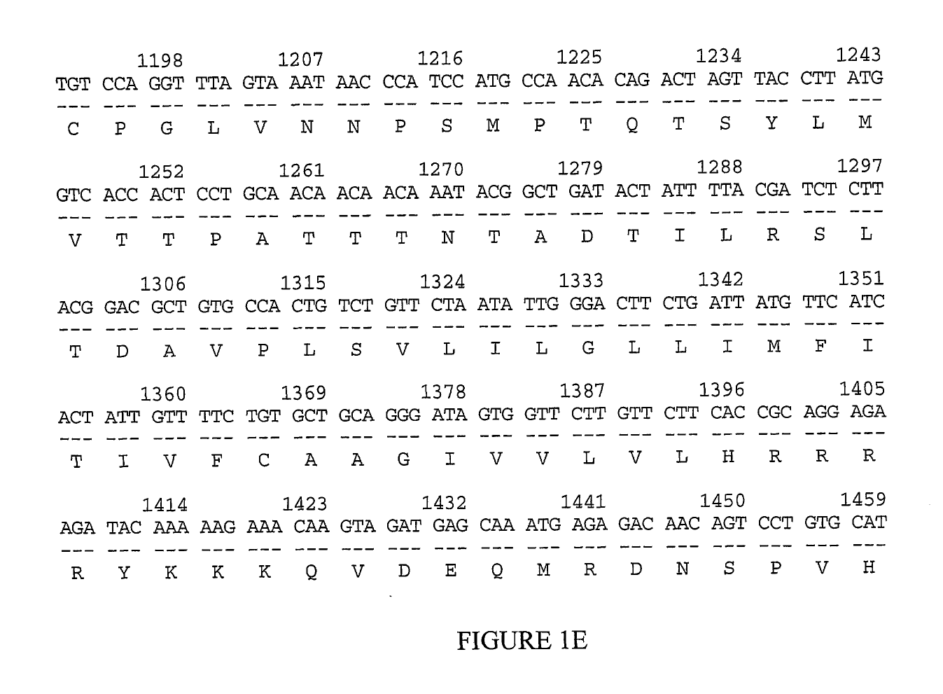

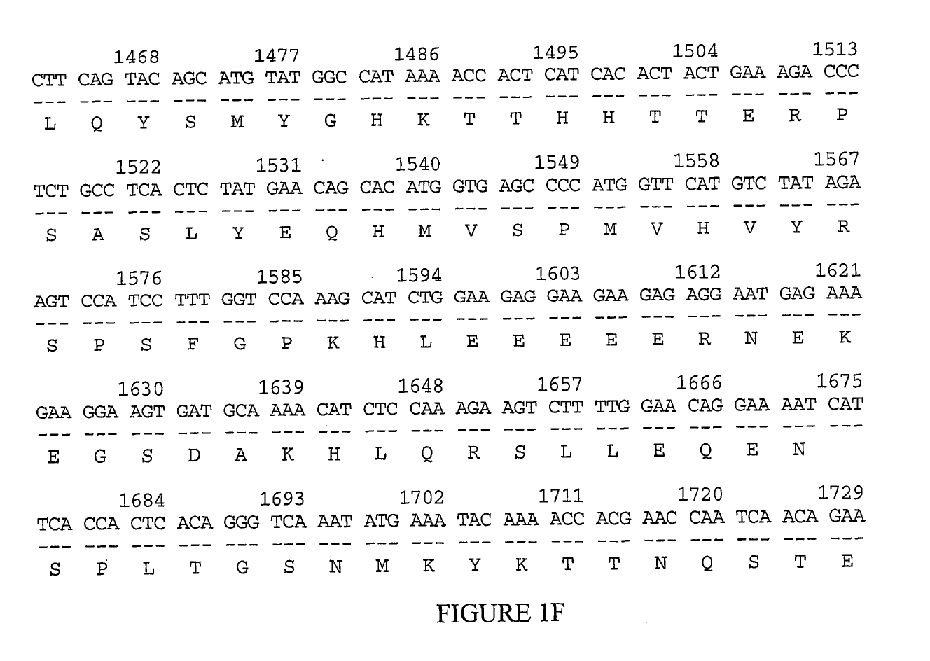

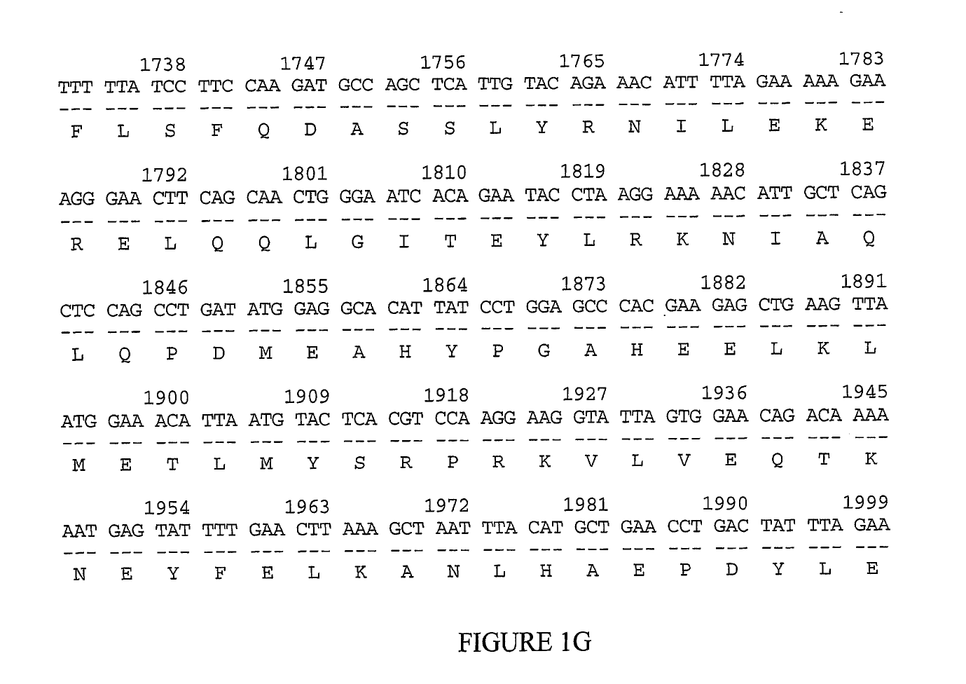

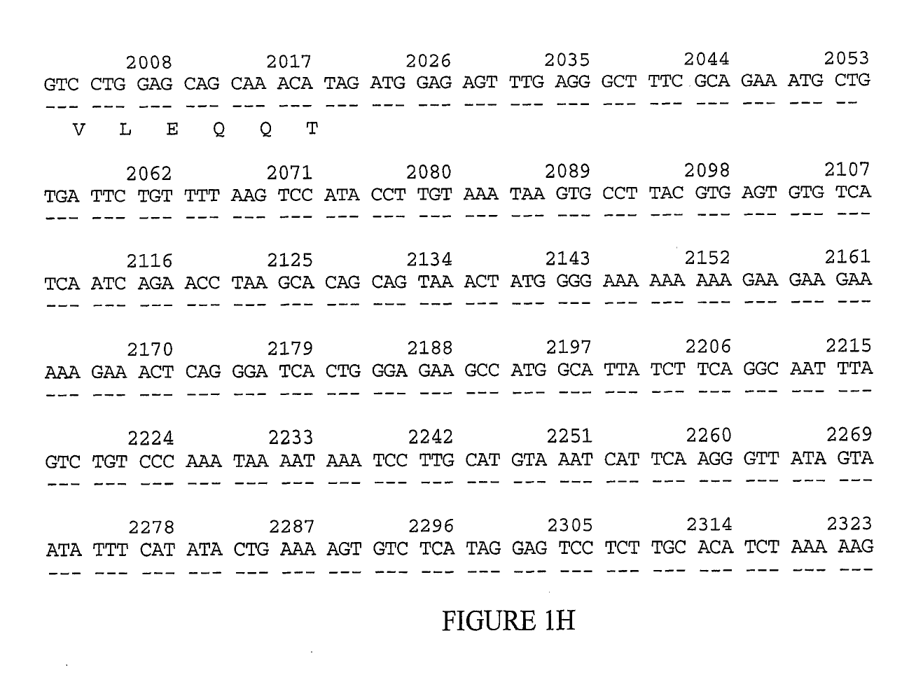

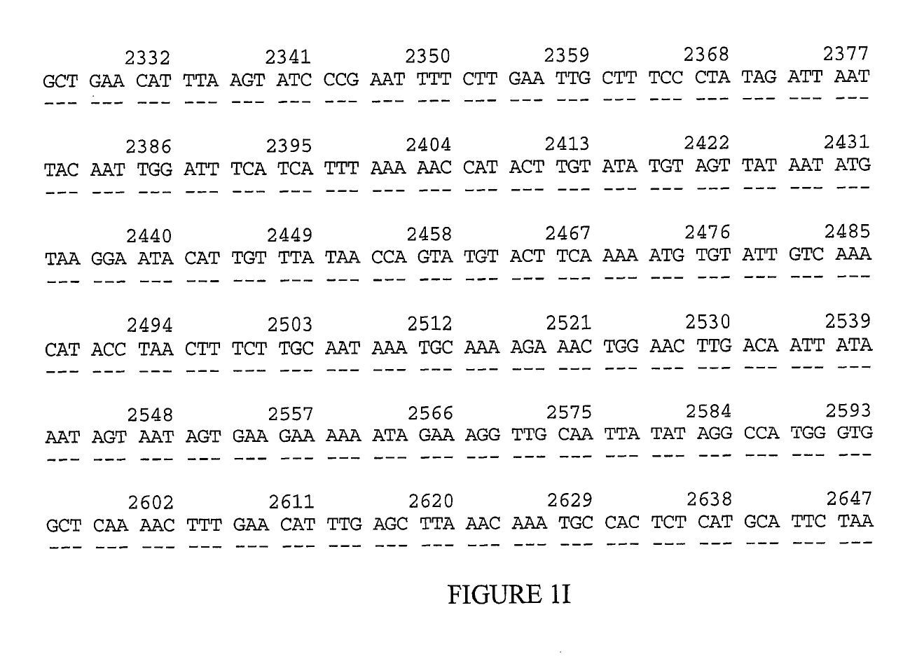

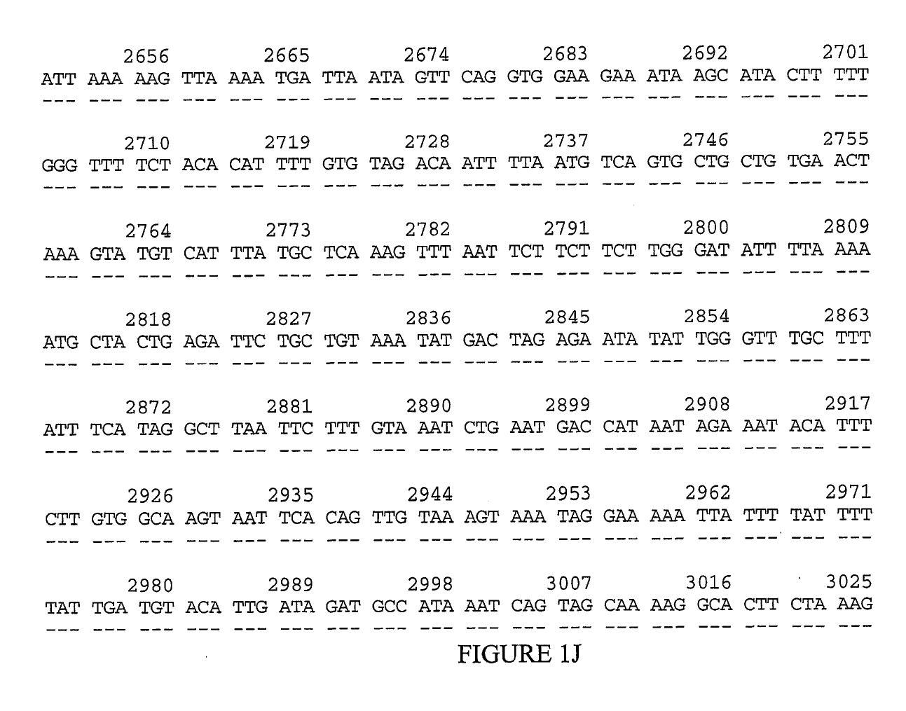

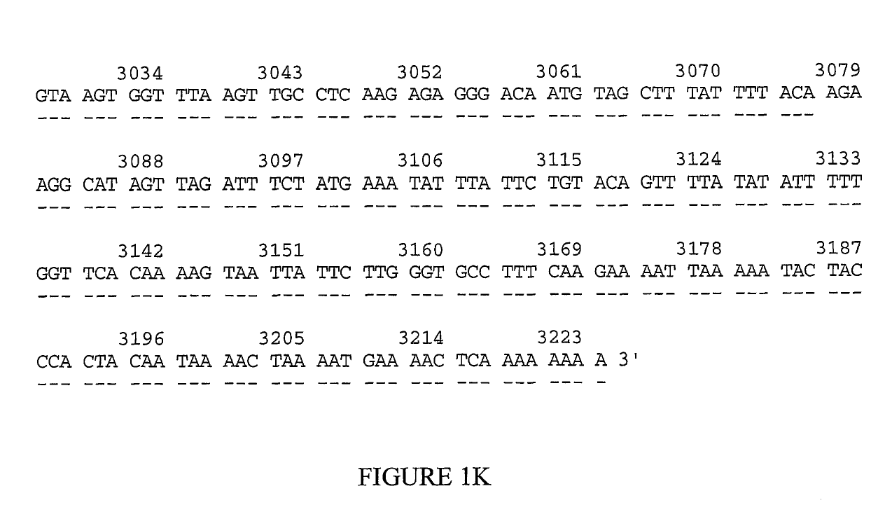

In one embodiment, the invention encompasses a polypeptide comprising the amino acid sequence of SEQ ID NO:1 as shown in FIGS. 1A through 1J. ECM-related protein is 546 amino acids in length and has two potential N-glycosylation sites at N114 and N446; eight potential casein kinase II phosphorylation sites at S59, T215, T244, S305, S381, S425, S453, and T521; two potential protein kinase C phosphorylation sites at S2 and T375; and one potential tyrosine kinase phosphorylation site at Y510. PFAM analysis indicates several leucine rich repeat domains (LRR) associated with protein-protein binding interactions between amino acid residues T93 and P188 of SEQ ID NO:1. PFAM analysis further indicates a C-terminal LLR from residues N222 through P272. PRINTS analysis further confirms the presence of LLRs from L118 to L131 and from M 163 to L176 of SEQ ID NO:1 HMMR analysis further indicates the presence of a transmembrane domain between residues 1316 and V335 of SEQ ID NO:1. A useful antigenic epitope extends from about L60 to about L175 of ECMRP and biologically active portions of ECMRP extend from about L118 to about L131, and from about M163 to about L176. An antibody which specifically binds ECM-related protein is useful in a diagnostic assay to identify a cancer, in particular color and lung cancer.

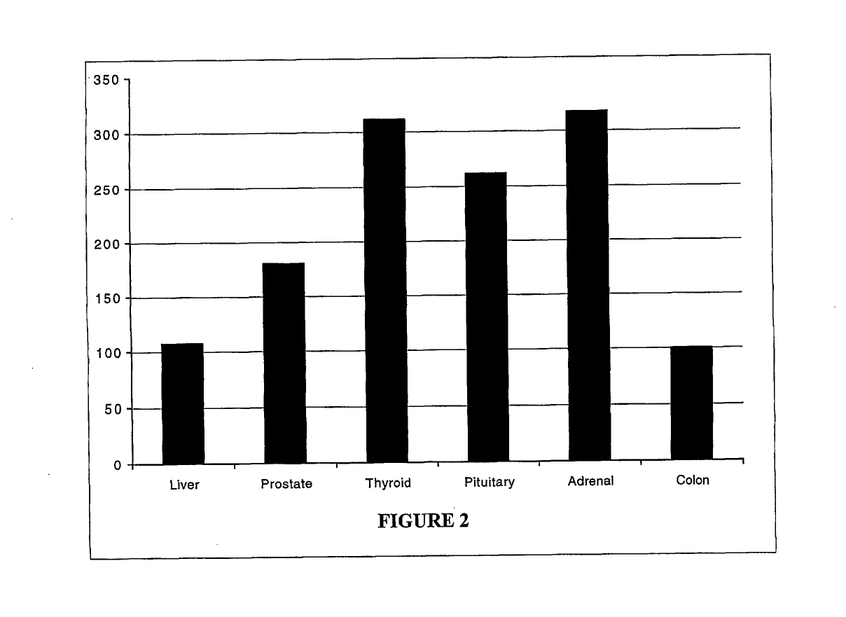

FIG. 2 shows the results of various normal adult tissues analyzed for ECMRP expression by TAQMAN analysis. Significant expression of the ECMRP was found only in liver, prostate, colon, thyroid, pituitary and adrenal tissues, and was undetectable in heart, brain, lung, skeletal muscle, kidney, pancreas, spleen, thymus, ovary, small intestine, and peripheral blood leukocytes.

Table 1 shows the expression of the ECMRP across tissue categories by Northern analysis of cDNA libraries in the LifeSeq database (Incyte Genomics). The results show the expression of ECMRP distributed across a variety of tissue categories including digestive system, endocrine and exocrine system, male genitalia (e.g., prostate), respiratory system, and skin. The differences observed between the results of Table 1 and FIG. 2, above, most likely reflect the high incidence of fetal and diseased tissues in cDNA libraries of the LifeSeq database.

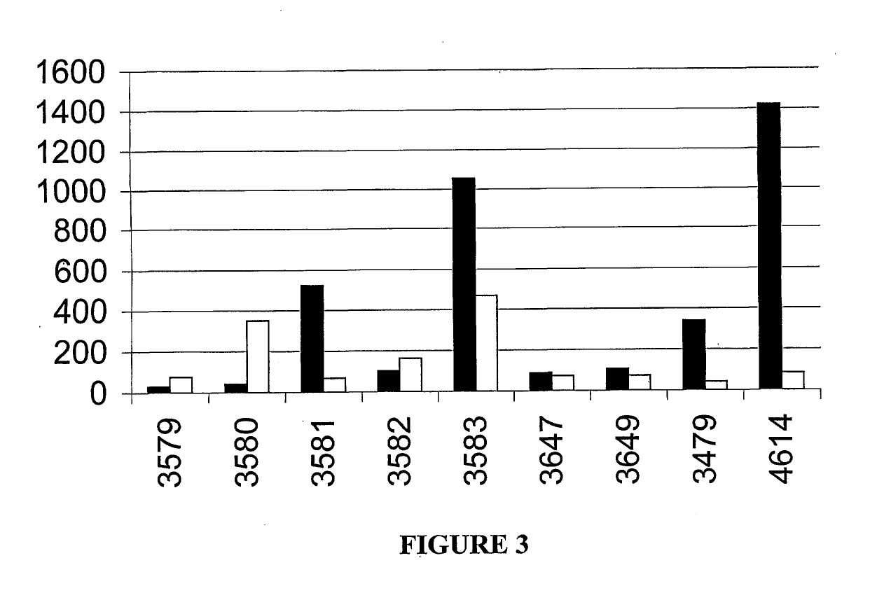

FIG. 3 shows the expression of ECMRP in colon cancer tissue samples compared with normal colon tissue and in and lung tumor samples compared with normal lung performed by TAQMAN analysis (Applied Biosystems). The results show an increased expression of ECMRP in colon tumors in 6 out of 9 samples examined (Donor IDs:3581, 3583, 3647, 3649, 3479, and 4614), The results were considered significant if at least a 1.2-fold difference in expression between cancerous and normal tissue was observed.

Table 2 shows the results of microarray analysis comparing the expression of ECMRP in colon cancer or colon polyp tissues relative to normal colon tissue, and in lung tumor tissue relative to normal lung. The results show an increased expression of ECMRP in colon tumors or polyps in 7 of 14 patients examined (Donor IDs:4614, 3755, 3311, 3754, 3583, 3839, and 3581. Increased expression of ECMRP was also observed in 2 of 10 lung tumor samples examined (Donor IDs:5796 and 5800). Differential expression (column 2) was considered significant if at least a 1.5-fold difference in expression between cancerous and normal tissue was observed.

Increased expression of ECMRP was also observed in a human colorectal adenocarcinoma cell line, HT-29, when compared to normal colon tissue. (Data not shown)

Mammalian variants of the cDNA encoding ECM-related protein were identified using BLAST2 with default parameters and the ZOOSEQ databases (Incyte Genomics). These preferred variants have from about 87% to 89% identity as shown in the table below. The first column, the SEQ IDvar for variant cDNAs; the third column, the clone number for the variant cDNAs; the fourth column, the percent identity to the human cDNA; and the fifth column, the alignment of the variant cDNA to the human cDNA.

| SEQ ID_{Var} | cDNA_{Var} | Species | Identity | Nt_{H} Alignment |
|---|---|---|---|---|
| 14 | 703545995J1 | Dog | 89% | 2-192 |
| 15 | 112357_Mm.1 | Monkey | 87% | 3003-3173 |

It will be appreciated by those skilled in the art that as a result of the degeneracy of the genetic code, a multitude of cDNAs encoding ECM-related protein , some bearing minimal similarity to the cDNAs of any known and naturally occurring gene, may be produced. Thus, the invention contemplates each and every possible variation of cDNA that could be made by selecting combinations based on possible codon choices. These combinations are made in accordance with the standard triplet genetic code as applied to the polynucleotide encoding naturally occurring ECM-related protein, and all such variations are to be considered as being specifically disclosed.

The cDNAs of SEQ ID NOs:2-15 may be used in hybridization, amplification, and screening technologies to identify and distinguish among SEQ ID NO:2 and related molecules in a sample. The mammalian cDNAs, SEQ ID NOs:14-15, may be used to produce transgenic cell lines or organisms which are model systems for human colon or lung cancer and upon which the toxicity and efficacy of potential therapeutic treatments may be tested. Toxicology studies, clinical trials, and subject/patient treatment profiles may be performed and monitored using the cDNAs, proteins, antibodies and molecules and compounds identified using the cDNAs and proteins of the present invention.

The identification and characterization of the cDNAs and proteins, fragments or portions thereof, were described in provisional application U.S. Serial No. 60/215,454, incorporated by reference herein in its entirety.

Characterization and Use of the Invention

cDNA Libraries

In a particular embodiment disclosed herein, MRNA is isolated from mammalian cells and tissues using methods which are well known to those skilled in the art and used to prepare the cDNA libraries. The Incyte cDNAs were isolated from mammalian cDNA libraries a prepared as described in the EXAMPLES. The consensus sequences are chemically and/or electronically assembled from fragments including Incyte cDNAs and extension and/or shotgun sequences using computer programs such as PHRAP (P Green, University of Washington, Seattle Wash.), and AUTOASSEMBLER application (Applied Biosystems, Foster City Calif.). After verification of the 5′ and 3′ sequence, at least one representative cDNA which encodes ECM-related protein is designated a reagent.

Sequencing

Methods for sequencing nucleic acids are well known in the art and may be used to practice any of the embodiments of the invention. These methods employ enzymes such as the Klenow fragment of DNA polymerase I, SEQUENASE, Taq DNA polymerase and thermostable T7 DNA polymerase (Amersham Pharmacia Biotech (APB), Piscataway N.J.), or combinations of polymerases and proofreading exonucleases such as those found in the ELONGASE amplification system (Life Technologies, Gaithersburg Md.). Preferably, sequence preparation is automated with machines such as the MICROLAB 2200 system (Hamilton, Reno Nev.) and the DNA ENGINE thermal cycler MJ Research, Watertown Mass.). Machines commonly used for sequencing include the ABI PRISM 3700, 377 or 373 DNA sequencing systems (Applied Biosystems), the MEGABACE 1000 DNA sequencing system (APB), and the like. The sequences may be analyzed using a variety of algorithms well known in the art and described in Ausubel et al. (1997; Short Protocols in Molecular Biology, John Wiley & Sons, New York N.Y., unit 7.7) and in Meyers (1995; Molecular Biology and Biotechnology, Wiley VCH, New York N.Y., pp. 856-853).

Shotgun sequencing may also be used to complete the sequence of a particular cloned insert of interest. Shotgun strategy involves randomly breaking the original insert into segments of various sizes and cloning these fragments into vectors. The fragments are sequenced and reassembled using overlapping ends until the entire sequence of the original insert is known. Shotgun sequencing methods are well known in the art and use thermostable DNA polymerases, heat-labile DNA polymerases, and primers chosen from representative regions flanking the cDNAs of interest. Incomplete assembled sequences are inspected for identity using various algorithms or programs such as CONSED (Gordon (1998) Genome Res 8:195-202) which are well known in the art. Contaminating sequences, including vector or chimeric sequences, or deleted sequences can be removed or restored, respectively, organizing the incomplete assembled sequences into finished sequences.

Extension of a Nucleic Acid Sequence

The sequences of the invention may be extended using various PCR-based methods known in the art. For example, the XL-PCR kit (Applied Biosystems), nested primers, and commercially available cDNA or genomic DNA libraries may be used to extend the nucleic acid sequence. For all PCR-based methods, primers may be designed using commercially available software, such as OLIGO primer analysis software (Molecular Biology Insights, Cascade Colo.) to be about 22 to 30 nucleotides in length, to have a GC content of about 50% or more, and to anneal to a target molecule at temperatures from about 55C to about 68C. When extending a sequence to recover regulatory elements, it is preferable to use genomic, rather than cDNA libraries.

Hybridization

The cDNA and fragments thereof can be used in hybridization technologies for various purposes. A probe may be designed or derived from unique regions such as the 5′ regulatory region or from a nonconserved region (i.e., 5′ or 3′ of the nucleotides encoding the conserved catalytic domain of the protein) and used in protocols to identify naturally occurring molecules encoding the ECM-related protein, allelic variants, or related molecules. The probe may be DNA or RNA, may be single-stranded, and should have at least 50% sequence identity to any of the nucleic acid sequences, SEQ ID NOs:2-15. Hybridization probes may be produced using oligolabeling, nick translation, end-labeling, or PCR amplification in the presence of a reporter molecule. A vector containing the cDNA or a fragment thereof may be used to produce an MRNA probe in vitro by addition of an RNA polymerase and labeled nucleotides. These procedures may be conducted using commercially available kits such as those provided by APB.

The stringency of hybridization is determined by G+C content of the probe, salt concentration, and temperature. In particular, stringency can be increased by reducing the concentration of salt or raising the hybridization temperature. Hybridization can be performed at low stringency with buffers, such as 5×SSC with 1% sodium dodecyl sulfate (SDS) at 60C, which permits the formation of a hybridization complex between nucleic acid sequences that contain some mismatches. Subsequent washes are performed at higher stringency with buffers such as 0.2×SSC with 0.1% SDS at either 45C (medium stringency) or 68C (high stringency). At high stringency, hybridization complexes will remain stable only where the nucleic acids are completely complementary. In some membrane-based hybridizations, preferably 35% or most preferably 50%, formamide can be added to the hybridization solution to reduce the temperature at which hybridization is performed, and background signals can be reduced by the use of detergents such as Sarkosyl or TRITON X-100 (Sigma-Aldrich, St. Louis Mo.) and a blocking agent such as denatured salmon sperm DNA. Selection of components and conditions for hybridization are well known to those skilled in the art and are reviewed in Ausubel (supra) and Sambrook et al. (1989) Molecular Cloning, A Laboratory Manual, Cold Spring Harbor Press, Plainview N.Y.

Arrays may be prepared and analyzed using methods well known in the art. Oligonucleotides or cDNAs may be used as hybridization probes or targets to monitor the expression level of large numbers of genes simultaneously or to identify genetic variants, mutations, and single nucleotide polymorphisms. Arrays may be used to determine gene function; to understand the genetic basis of a condition, disease, or disorder; to diagnose a condition, disease, or disorder; and to develop and monitor the activities of therapeutic agents. (See, e.g., Brennan et al. (1995) U.S. Pat. No. 5,474,796; Schena et al. (1996) Proc Natl Acad Sci 93:10614-10619; Heller et al. (1997) Proc Natl Acad Sci 94:2150-2155; and Heller et al. (1997) U.S. Pat. No. 5,605,662.)

Hybridization probes are also useful in mapping the naturally occurring genomic sequence. The probes may be hybridized to a particular chromosome, a specific region of a chromosome, or an artificial chromosome construction. Such constructions include human artificial chromosomes (HAC), yeast artificial chromosomes (YAC), bacterial artificial chromosomes (BAC), bacterial P1 constructions, or the cDNAs of libraries made from single chromosomes.

Expression

Any one of a multitude of cDNAs encoding ECM-related protein may be cloned into a vector and used to express the protein, or portions thereof, in host cells. The nucleic acid sequence can be engineered by such methods as DNA shuffling (U.S. Pat. No. 5,830,721) and site-directed mutagenesis to create new restriction sites, alter glycosylation patterns, change codon preference to increase expression in a particular host, produce splice variants, extend half-life, and the like. The expression vector may contain transcriptional and translational control elements (promoters, enhancers, specific initiation signals, and polyadenylated 3′ sequence) from various sources which have been selected for their efficiency in a particular host. The vector, cDNA, and regulatory elements are combined using in vitro recombinant DNA techniques, synthetic techniques, and/or in vivo genetic recombination techniques well known in the art and described in Sambrook (supra, ch. 4, 8, 16 and 17).

A variety of host systems may be transformed with an expression vector. These include, but are not limited to, bacteria transformed with recombinant bacteriophage, plasmid, or cosmid DNA expression vectors; yeast transformed with yeast expression vectors; insect cell systems transformed with baculovirus expression vectors; plant cell systems transformed with expression vectors containing viral and/or bacterial elements, or animal cell systems (Ausubel supra, unit 16). For example, an adenovirus transcription/translation complex may be utilized in mammalian cells. After sequences are ligated into the E1 or E3 region of the viral genome, the infective virus is used to transform and express the protein in host cells. The Rous sarcoma virus enhancer or SV40 or EBV-based vectors may also be used for high-level protein expression.

Routine cloning, subcloning, and propagation of nucleic acid sequences can be achieved using the multifunctional PBLUESCRIPT vector (Stratagene, La Jolla Calif.) or PSPORT1 plasmid (Life Technologies). Introduction of a nucleic acid sequence into the multiple cloning site of these vectors disrupts the lacZ gene and allows colorimetric screening for transformed bacteria. In addition, these vectors may be useful for in vitro transcription, dideoxy sequencing, single strand rescue with helper phage, and creation of nested deletions in the cloned sequence.

For long term production of recombinant proteins, the vector can be stably transformed into cell lines along with a selectable or visible marker gene on the same or on a separate vector. After transformation, cells are allowed to grow for about 1 to 2 days in enriched media and then are transferred to selective media. Selectable markers, antimetabolite, antibiotic, or herbicide resistance genes, confer resistance to the relevant selective agent and allow growth and recovery of cells which successfully express the introduced sequences. Resistant clones identified either by survival on selective media or by the expression of visible markers may be propagated using culture techniques. Visible markers are also used to estimate the amount of protein expressed by the introduced genes. Verification that the host cell contains the desired cDNA is based on DNA-DNA or DNA-RNA hybridizations or PCR amplification techniques.

The host cell may be chosen for its ability to modify a recombinant protein in a desired fashion. Such modifications include acetylation, carboxylation, glycosylation, phosphorylation, lipidation, acylation and the like. Post-translational processing which cleaves a “prepro” form may also be used to specify protein targeting, folding, and/or activity. Different host cells available from the ATCC (Manassas Va.) which have specific cellular machinery and characteristic mechanisms for post-translational activities may be chosen to ensure the correct modification and processing of the recombinant protein.

Recovery of Proteins from Cell Culture

Heterologous moieties engineered into a vector for ease of purification include glutathione S-transferase (GST), 6×His, FLAG, MYC, and the like. GST and 6-His are purified using commercially available affinity matrices such as immobilized glutathione and metal-chelate resins, respectively. FLAG and MYC are purified using commercially available monoclonal and polyclonal antibodies. For ease of separation following purification, a sequence encoding a proteolytic cleavage site may be part of the vector located between the protein and the heterologous moiety. Methods for recombinant protein expression and purification are discussed in Ausubel (supra, unit 16) and are commercially available.

Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds α-amino- and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-α-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivitized amino acids, and washing with dichloromethane and/or N,N-dimethylformamide. The peptide is cleaved between the peptide carboxy terminus and the linker group to yield a peptide acid or amide. (Novabiochem 1997/98 Catalog and Peptide Synthesis Handbook, San Diego Calif. pp. S1-S20). Automated synthesis may also be carried out on machines such as the ABI 431A peptide synthesizer (Applied Biosystems). A protein or portion thereof may be purified by preparative high performance liquid chromatography and its composition confirmed by amino acid analysis or by sequencing (Creighton (1984) Proteins, Structures and Molecular Properties, W H Freeman, New York N.Y.).

Preparation and Screening of Antibodies

Various hosts including, but not limited to, goats, rabbits, rats, mice, and human cell lines may be immunized by injection with ECM-related protein or any portion thereof. Adjuvants such as Freund's, mineral gels, and surface active substances such as lysolecithin, pluronic polyols, polyanions, peptides, oil emulsions, keyhole limpet hemacyanin (KLH), and dinitrophenol may be used to increase immunological response. The oligopeptide, peptide, or portion of protein used to induce antibodies should consist of at least about five amino acids, more preferably ten amino acids, which are identical to a portion of the natural protein. Oligopeptides may be fused with proteins such as KLH in order to produce antibodies to the chimeric molecule.

Monoclonal antibodies may be prepared using any technique which provides for the production of antibodies by continuous cell lines in culture. These include, but are not limited to, the hybridoma technique, the human B-cell hybridoma technique, and the EBV-hybridoma technique. (See, e.g., Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120.)

Alternatively, techniques described for antibody production may be adapted, using methods known in the art, to produce epitope-specific, single chain antibodies. Antibody fragments which contain specific binding sites for epitopes of the protein may also be generated. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (See, e.g., Huse et al. (1989) Science 246:1275-1281.)

The ECM-related protein or a portion thereof may be used in screening assays of phagemid or B-lymphocyte immunoglobulin libraries to identify antibodies having the desired specificity. Numerous protocols for competitive binding or immunoassays using either polyclonal or monoclonal antibodies with established specificities are well known in the art. Such immunoassays typically involve the measurement of complex formation between the protein and its specific antibody. A two-site, monoclonal-based immunoassay utilizing monoclonal antibodies reactive to two non-interfering epitopes is preferred, but a competitive binding assay may also be employed (Pound (1998) Immunochemical Protocols, Humana Press, Totowa N.J.).

Various methods such as Scatchard analysis in conjunction with radioimmunoassay techniques may be used to assess the affinity of antibodies for ECMRP. Affinity is expressed as an association constant, Kₐ, which is defined as the molar concentration of HSPDE10A-antibody complex divided by the molar concentrations of free antigen and free antibody under equilibrium conditions. The Kₐ determined for a preparation of polyclonal antibodies, which are heterogeneous in their affinities for multiple epitopes, represents the average affinity, or avidity, of the antibodies for ECMRP. The Kₐ determined for a preparation of monoclonal antibodies, which are monospecific for a particular epitope, represents a true measure of affinity. High-affinity antibody preparations with Kₐ ranging from about 10⁹ to 10¹² l/mole are preferred for use in immunoassays in which the protein-antibody complex must withstand rigorous manipulations. Low-affinity antibody preparations with Kₐ ranging from about 10⁶ to 10⁷ l/mole are preferred for use in immunopurification and similar procedures which ultimately require dissociation of ECMRP, preferably in active form, from the antibody (Catty (1988) Antibodies, Volume I: A Practical Approach, IRL Press, Washington D.C.; Liddell and Cryer (1991) A Practical Guide to Monoclonal Antibodies, John Wiley, New York N.Y.).

Labeling of Molecules for Assay

A wide variety of reporter molecules and conjugation techniques are known by those skilled in the art and may be used in various nucleic acid, amino acid, and antibody assays. Synthesis of labeled molecules may be achieved using commercially available kits (Promega, Madison Wis.) for incorporation of a labeled nucleotide such as ³²P-dCTP (APB), Cy3-dCTP or Cy5-dCTP (Operon Technologies, Alameda Calif.), or amino acid such as ³⁵S-methionine (APB). Nucleotides and amino acids may be directly labeled with a variety of substances including fluorescent, chemiluminescent, or chromogenic agents, and the like, by chemical conjugation to amines, thiols and other groups present in the molecules using reagents such as BIODIPY or FITC (Molecular Probes, Eugene Oreg.).

Diagnostics

Nucleic Acid Assays

The cDNAs, fragments, oligonucleotides, complementary RNA and DNA molecules, and PNAs and may be used to detect and quantify differential gene expression for diagnosis of a disorder. Similarly antibodies which specifically bind ECM-related protein may be used to quantitate the protein. Disorders associated with differential expression include cancer, in particular, colon and lung cancer. The diagnostic assay may use hybridization or amplification technology to compare gene expression in a biological sample from a patient to standard samples in order to detect differential gene expression. Qualitative or quantitative methods for this comparison are well known in the art.

For example, the cDNA or probe may be labeled by standard methods and added to a biological sample from a patient under conditions for the formation of hybridization complexes. After an incubation period, the sample is washed and the amount of label (or signal) associated with hybridization complexes, is quantified and compared with a standard value. If complex formation in the patient sample is significantly altered (higher or lower) in comparison to either a normal or disease standard, then differential expression indicates the presence of a disorder.

In order to provide standards for establishing differential expression, normal and disease expression profiles are established. This is accomplished by combining a sample taken from normal subjects, either animal or human, with a cDNA under conditions for hybridization to occur. Standard hybridization complexes may be quantified by comparing the values obtained using normal subjects with values from an experiment in which a known amount of a purified sequence is used. Standard values obtained in this manner may be compared with values obtained from samples from patients who were diagnosed with a particular condition, disease, or disorder. Deviation from standard values toward those associated with a particular disorder is used to diagnose that disorder.

Such assays may also be used to evaluate the efficacy of a particular therapeutic treatment regimen in animal studies or in clinical trials or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to years.

Protein Assays

Detection and quantification of a protein using either labeled amino acids or specific polyclonal or monoclonal antibodies are known in the art. Examples of such techniques include two-dimensional polyacrylamide gel electrophoresis, enzyme-linked immunosorbent assays (ELISAs), radioimmunoassays (RIAs), and fluorescence activated cell sorting (FACS). These assays and their quantitation against purifed, labeled standards are well known in the art (Ausubel, supra, unit 10.1-10.6). A two-site, monoclonal-based immunoassay utilizing monoclonal antibodies reactive to two non-interfering epitopes is preferred, but a competitive binding assay may be employed. (See, e.g., Coligan et at. (1997) Current Protocols in Immunology, Wiley-Interscience, New York N.Y.; and Pound, supra.)

Therapeutics

Chemical and structural similarity, in particular the a transmembrane domain and several LLR domains, exists between regions of ECM-related protein (SEQ ID NO:1) and other ECM-related proteins. In addition, differential expression is highly associated with colon and lung cancer as shown in Table 2 and FIG. 3. ECM-related protein clearly plays a role in cancer, in particular, colon and lung cancer.

In the treatment of conditions associated with increased expression of the protein such as colon or lung cancer, it is desirable to decrease expression or protein activity. In one embodiment, the an inhibitor, antagonist or antibody of the protein may be administered to a subject to treat a condition associated with increased expression or activity. In another embodiment, a pharmaceutical composition comprising an inhibitor, antagonist, or antibody and a pharmaceutical carrier may be administered to a subject to treat a condition associated with the increased expression or activity of the endogenous protein. In an additional embodiment, a vector expressing the complement of the cDNA or fragments thereof may be administered to a subject to treat the disorder.

Any of the cDNAs, complementary molecules, or fragments thereof, proteins or portions thereof, vectors delivering these nucleic acid molecules or expressing the proteins, and their ligands may be administered in combination with other therapeutic agents. Selection of the agents for use in combination therapy may be made by one of ordinary skill in the art according to conventional pharmaceutical principles. A combination of therapeutic agents may act synergistically to affect treatment of a particular disorder at a lower dosage of each agent.

Modification of Gene Expression Using Nucleic Acids

Gene expression may be modified by designing complementary or antisense molecules (DNA, RNA, or PNA) to the control, 5′, 3′, or other regulatory regions of the gene encoding ECM-related protein. Oligonucleotides designed to inhibit transcription initiation are preferred. Similarly, inhibition can be achieved using triple helix base-pairing which inhibits the binding of polymerases, transcription factors, or regulatory molecules (Gee et al. In: Huber and Carr (1994) Molecular and Immunologic Approaches, Futura Publishing, Mt. Kisco N.Y., pp. 163-177). A complementary molecule may also be designed to block translation by preventing binding between ribosomes and mRNA. In one alternative, a library or plurality of cDNAs may be screened to identify those which specifically bind a regulatory, nontranslated sequence.

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage at sites such as GUA, GUU, and GUC. Once such sites are identified, an oligonucleotide with the same sequence may be evaluated for secondary structural features which would render the oligonucleotide inoperable. The suitability of candidate targets may also be evaluated by testing their hybridization with complementary oligonucleotides using ribonuclease protection assays.

Complementary nucleic acids and ribozymes of the invention may be prepared via recombinant expression, in vitro or in vivo, or using solid phase phosphoramidite chemical synthesis. In addition, RNA molecules may be modified to increase intracellular stability and half-life by addition of flanking sequences at the 5′ and/or 3′ ends of the molecule or by the use of phosphorothioate or 2′ O-methyl rather than phosphodiesterase linkages within the backbone of the molecule. Modification is inherent in the production of PNAs and can be extended to other nucleic acid molecules. Either the inclusion of nontraditional bases such as inosine, queosine, and wybutosine, or the modification of adenine, cytidine, guanine, thymine, and uridine with acetyl-, methyl-, thio-groups renders the molecule less available to endogenous endonucleases.

Screening and Purification Assays

The cDNA encoding ECM-related protein may be used to screen a library or a plurality of molecules or compounds for specific binding affinity. The libraries may be DNA molecules, RNA molecules, PNAs, peptides, proteins such as transcription factors, enhancers, or repressors, and other ligands which regulate the activity, replication, transcription, or translation of the endogenous gene. The assay involves combining a polynucleotide with a library or plurality of molecules or compounds under conditions allowing specific binding, and detecting specific binding to identify at least one molecule which specifically binds the single-stranded or double-stranded molecule.

In one embodiment, the cDNA of the invention may be incubated with a plurality of purified molecules or compounds and binding activity determined by methods well known in the art, e.g., a gel-retardation assay (U.S. Pat. No. 6,010,849) or a reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay and may be later confirmed by recovering and raising antibodies against that molecule or compound. When these antibodies are added into the assay, they cause a supershift in the gel-retardation assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

In a further embodiment, the protein or a portion thereof may be used to purify a ligand from a sample. A method for using a protein or a portion thereof to purify a ligand would involve combining the protein or a portion thereof with a sample under conditions to allow specific binding, detecting specific binding between the protein and ligand, recovering the bound protein, and using a chaotropic agent to separate the protein from the purified ligand.

In a preferred embodiment, ECM-related protein may be used to screen a plurality of molecules or compounds in any of a variety of screening assays. The portion of the protein employed in such screening may be free in solution, affixed to an abiotic or biotic substrate (e.g. borne on a cell surface), or located intracellularly. For example, in one method, viable or fixed prokaryotic host cells that are stably transformed with recombinant nucleic acids that have expressed and positioned a peptide on their cell surface can be used in screening assays. The cells are screened against a plurality or libraries of ligands, and the specificity of binding or formation of complexes between the expressed protein and the ligand can be measured. Depending on the particular kind of molecules or compounds being screened, the assay may be used to identify DNA molecules, RNA molecules, peptide nucleic acids, peptides, proteins, mimetics, agonists, antagonists, antibodies, immunoglobulins, inhibitors, and drugs or any other ligand, which specifically binds the protein.

In one aspect, this invention comtemplates a method for high throughput screening using very small assay volumes and very small amounts of test compound as described in U.S. Pat. No. 5,876,946, incorporated herein by reference. This method is used to screen large numbers of molecules and compounds via specific binding. In another aspect, this invention also contemplates the use of competitive drug screening assays in which neutralizing antibodies capable of binding the protein specifically compete with a test compound capable of binding to the protein. Molecules or compounds identified by screening may be used in a mammalian model system to evaluate their toxicity, diagnostic, or therapeutic potential.

Pharmacology

Pharmaceutical compositions contain active ingredients in an effective amount to achieve a desired and intended purpose and a pharmaceutical carrier. The determination of an effective dose is well within the capability of those skilled in the art. For any compound, the therapeutically effective dose may be estimated initially either in cell culture assays or in animal models. The animal model is also used to achieve a desirable concentration range and route of administration. Such information may then be used to determine useful doses and routes for administration in humans.

A therapeutically effective dose refers to that amount of protein or inhibitor which ameliorates the symptoms or condition. Therapeutic efficacy and toxicity of such agents may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it may be expressed as the ratio, LD₅₀/ED₅₀. Pharmaceutical compositions which exhibit large therapeutic indexes are preferred. The data obtained from cell culture assays and animal studies are used in formulating a range of dosage for human use.

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of under- or over-expression of genes of interest and for the development of methods for diagnosis and treatment of diseases. A mammal inbred to over-express a particular gene (for example, secreted in milk) may also serve as a convenient source of the protein expressed by that gene.

Toxicology

Toxicology is the study of the effects of agents on living systems. The majority of toxicity studies are performed on rats or mice. Observation of qualitative and quantitative changes in physiology, behavior, homeostatic processes, and lethality in the rats or mice are used to generate a toxicity profile and to assess potential consequences on human health following exposure to the agent.

Genetic toxicology identifies and analyzes the effect of an agent on the rate of endogenous, spontaneous, and induced genetic mutations. Genotoxic agents usually have common chemical or physical properties that facilitate interaction with nucleic acids and are most harmful when chromosomal aberrations are transmitted to progeny. Toxicological studies may identify agents that increase the frequency of structural or functional abnormalities in the tissues of the progeny if administered to either parent before conception, to the mother during pregnancy, or to the developing organism. Mice and rats are most frequently used in these tests because their short reproductive cycle allows the production of the numbers of organisms needed to satisfy statistical requirements.

Acute toxicity tests are based on a single administration of an agent to the subject to determine the symptomology or lethality of the agent. Three experiments are conducted: 1) an initial dose-range-finding experiment, 2) an experiment to narrow the range of effective doses, and 3) a final experiment for establishing the dose-response curve.

Subchronic toxicity tests are based on the repeated administration of an agent. Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, with a duration of a year or more, are used to demonstrate either the absence of toxicity or the carcinogenic potential of an agent. When studies are conducted on rats, a minimum of three test groups plus one control group are used, and animals are examined and monitored at the outset and at intervals throughout the experiment.

Transgenic Animal Models

Transgenic rodents that over-express or under-express a gene of interest may be inbred and used to model human diseases or to test therapeutic or toxic agents. (See, e.g., U.S. Pat. Nos. 5,175,383 and 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the potential to form embryonic tissues. When ES cells are placed inside a carrier embryo, they resume normal development and contribute to tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gen, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodermal cell types which differentiate into, for example, neural cells, hematopoietic lineages, and cardiomyocytes.

Knockout Analysis

In gene knockout analysis, a region of a mammalian gene is enzymatically modified to include a non-mammalian gene such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines which lack a functional copy of the mammalian gene. In one example, the mammalian gene is a human gene.

Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with potential pharmaceutical agents to obtain information on treatment of the analogous human condition. These methods have been used to model several human diseases.

Non-Human Primate Model

The field of animal testing deals with data and methodology from basic sciences such as physiology, genetics, chemistry, pharmacology and statistics. These data are paramount in evaluating the effects of therapeutic agents on non-human primates as they can be related to human health. Monkeys are used as human surrogates in vaccine and drug evaluations, and their responses are relevant to human exposures under similar conditions. Cynomolgus and Rhesus monkeys (Macaca fascicularis and Macaca mulatta, respectively) and Common Marmosets (Callithrix jacchus) are the most common non-human primates (NHPs) used in these investigations. Since great cost is associated with developing and maintaining a colony of NHPs, early research and toxicological studies are usually carried out in rodent models. In studies using behavioral measures such as drug addiction, NHPs are the first choice test animal. In addition, NHPs and individual humans exhibit differential sensitivities to many drugs and toxins and can be classified as a range of phenotypes from “extensive metabolizers” to “poor metabolizers” of these agents.

In additional embodiments, the cDNAs which encode the protein may be used in any molecular biology techniques that have yet to be developed, provided the new techniques rely on properties of cDNAs that are currently known, including, but not limited to, such properties as the triplet genetic code and specific base pair interactions.

### EXAMPLES

The examples below are provided to illustrate the subject invention and are not included for the purpose of limiting the invention. The preparation of the human colon polyp library, COLDNOT01, is described.

I cDNA Library Construction

COLDNOT01

The COLDNOT01 library was constructed using RNA isolated from diseased descending colon tissues from a 16-year-old male during partial colectomy, temporary ileostomy, and colonoscopy. Pathology indicated innumerable (greater than 100) adenomatous polyps with low grade dysplasia involving the entire colonic mucosa in the setting of familial polyposis coli.

The frozen tissue was homogenized and lysed in guanidinium isothiocyanate solution using a POLYTRON homogenizer (Brinkmann Instruments, Westbury N.J.). The lysate was centrifuged over a 5.7 M CsCl cushion using an SW28 rotor in an L8-70M ultracentrifuge (Beckman Coulter, Fullerton Calif.) for 18 hours at 25,000 rpm at ambient temperature. The RNA was extracted with acid phenol, pH 4.7, precipitated using 0.3 M sodium acetate and 2.5 volumes of ethanol, resuspended in RNAse-free water, and DNAse treated at 37° C. Extraction with acid phenol, pH 4.7, and precipitation with sodium acetate and ethanol was repeated. The mRNA was isolated with the OLIGOTEX kit (Qiagen, Chatsworth Calif.) and used to construct the cDNA library.

The mRNA was handled according to the recommended protocols in the SUPERSCRIPT plasmid system (Life Technologies) which contains a NotI primer-adaptor designed to prime the first strand cDNA synthesis at the poly(A) tail of mRNAs. Double stranded cDNA was blunted, ligated to EcoRI adaptors and digested with NotI (New England Biolabs, Beverly Mass.). The cDNAs were fractionated on a SEPHAROSE CL4B column (APB), and those cDNAs exceeding 400 bp were ligated into pINCY plasmid (Incyte Genomics). The plasmid pINCY was subsequently transformed into DH5α competent cells (Life Technologies).

II Construction of pINCY Plasmid

The plasmid was constructed by digesting the pSPORT1 plasmid (Life Technologies) with EcoRI restriction enzyme (New England Biolabs, Beverly Mass.) and filling the overhanging ends using Klenow enzyme (New England Biolabs) and 2′-deoxynucleotide 5′-triphosphates (dNTPs). The plasmid was self-ligated and transformed into the bacterial host, E. coli strain JM109.

An intermediate plasmid, pSPORT 1-ΔRI, which showed no digestion with EcoRI, was digested with Hind III (New England Biolabs); and the overhanging ends were filled in with Klenow and dNTPs. A linker sequence was phosphorylated, ligated onto the 5′ blunt end, digested with EcoRI, and self-ligated. Following transformation into JM109 host cells, plasmids were isolated and tested for preferential digestibility with EcoRI, but not with Hind III. A single colony that met this criteria was designated pINCY plasmid.

After testing the plasmid for its ability to incorporate cDNAs from a library prepared using NotI and EcoRI restriction enzymes, several clones were sequenced; and a single clone containing an insert of approximately 0.8 kb was selected from which to prepare a large quantity of the plasmid. After digestion with NotI and EcoRI, the plasmid was isolated on an agarose gel and purified using a QIAQUICK column (Qiagen) for use in library construction.

III Isolation and Sequencing of cDNA Clones

Plasmid DNA was released from the cells and purified using either the MINIPREP kit (Edge Biosystems, Gaithersburg Md.) or the REAL PREP 96 plasmid kit (Qiagen). A kit consists of a 96-well block with reagents for 960 purifications. The recommended protocol was employed except for the following changes: 1) the bacteria were cultured in 1 ml of sterile TERRIFIC BROTH (BD Biosciences, Sparks Md.) with carbenicillin at 25 mg/l and glycerol at 0.4%; 2) after inoculation, the cells were cultured for 19 hours and then lysed with 0.3 ml of lysis buffer; and 3) following isopropanol precipitation, the plasmid DNA pellet was resuspended in 0.1 ml of distilled water. After the last step in the protocol, samples were transferred to a 96-well block for storage at 4C.

The cDNAs were prepared for sequencing using the MICROLAB 2200 system (Hamilton) in combination with the DNA ENGINE thermal cyclers (MJ Research). The cDNAs were sequenced by the method of Sanger and Coulson (1975; J Mol Biol 94:441-448) using an ABI PRISM 377 sequencing system (Applied Biosystems) or the MEGABACE 1000 DNA sequencing system (APB). Most of the isolates were sequenced according to standard ABI protocols and kits (Applied Biosystems) with solution volumes of 0.25×-1.0× concentrations. In the alternative, cDNAs were sequenced using solutions and dyes from APB.

IV Extension of cDNA Sequences

The cDNAs were extended using the cDNA clone and oligonucleotide primers. One primer was synthesized to initiate 5′ extension of the known fragment, and the other, to initiate 3′ extension of the known fragment. The initial primers were designed using commercially available primer analysis software to be about 22 to 30 nucleotides in length, to have a GC content of about 50% or more, and to anneal to the target sequence at temperatures of about 68C to about 72C. Any stretch of nucleotides that would result in hairpin structures and primer-primer dimerizations was avoided.

Selected cDNA libraries were used as templates to extend the sequence. If more than one extension was necessary, additional or nested sets of primers were designed. Preferred libraries have been size-selected to include larger cDNAs and random primed to contain more sequences with 5′ or upstream regions of genes. Genomic libraries are used to obtain regulatory elements, especially extension into the 5′ promoter binding region.

High fidelity amplification was obtained by PCR using methods such as that taught in U.S. Pat. No. 5,932,451. PCR was performed in 96-well plates using the DNA ENGIE thermal cycler (MJ Research). The reaction mix contained DNA template, 200 nmol of each primer, reaction buffer containing Mg²⁺, (NH₄)₂SO₄, and β-mercaptoethanol, Taq DNA polymerase (APB), ELONGASE enzyme (Life Technologies), and Pfu DNA polymerase (Stratagene), with the following parameters for primer pair PCI A and PCI B (Incyte Genomics): Step 1: 94C, three min; Step 2: 94C, 15 sec; Step 3: 60C, one min; Step 4: 68C, two min; Step 5: Steps 2, 3, and 4 repeated 20 times; Step 6: 68C; five min; Step 7: storage at 4C. In the alternative, the parameters for primer pair T7 and SK+ (Stratagene) were as follows: Step 1: 94C, three min; Step 2: 94C, 15 sec; Step 3: 57C, one min; Step 4: 68C, two min; Step 5: Steps 2, 3, and 4 repeated 20 times; Step 6: 68C, five min; Step 7: storage at 4C.

The concentration of DNA in each well was determined by dispensing 100 MI PICOGREEN quantitation reagent (0.25% reagent in 1×TE, v/v; Molecular Probes) and 0.5 μl of undiluted PCR product into each well of an opaque fluorimeter plate (Corning, Acton Mass.) and allowing the DNA to bind to the reagent. The plate was scanned in a Fluoroskan II (Labsystems Oy) to measure the fluorescence of the sample and to quantify the concentration of DNA. A 5 μl to 10 μl aliquot of the reaction mixture was analyzed by electrophoresis on a 1% agarose minigel to determine which reactions were successful in extending the sequence.

The extended clones were desalted, concentrated, transferred to 384-well plates, digested with CviJI cholera virus endonuclease (Molecular Biology Research, Madison Wis.), and sonicated or sheared prior to religation into pUC18 vector (APB). For shotgun sequences, the digested nucleotide sequences were separated on low concentration (0.6 to 0.8%) agarose gels, fragments were excised, and the agar was digested with AGARACE enzyme (Promega). Extended clones were religated using T4 DNA ligase (New England Biolabs) into pUC18 vector (APB), treated with Pfu DNA polymerase (Stratagene) to fill-in restriction site overhangs, and transfected into E. coli competent cells. Transformed cells were selected on antibiotic-containing media, and individual colonies were picked and cultured overnight at 37C in 384-well plates in LB/2× carbenicillin liquid media.

The cells were lysed, and DNA was amplified using primers, Taq DNA polymerase (APB) and Pfu DNA polymerase (Stratagene) with the following parameters: Step 1: 94C, three min; Step 2: 94C, 15 sec; Step 3: 60C, one min; Step 4: 72C, two min; Step 5: steps 2, 3, and 4 repeated 29 times; Step 6: 72C, five min; Step 7: storage at 4C. DNA was quantified using PICOGREEN quantitation reagent (Molecular Probes) as described above. Samples with low DNA recoveries were reamplified using the conditions described above. Samples were diluted with 20% dimethylsulfoxide (DMSO; 1:2, v/v), and sequenced using DYENAMIC energy transfer sequencing primers and the DYENAMIC DIRECT cycle sequencing kit (APB) or the ABI PRISM BIGDYE terminator cycle sequencing kit (Applied Biosystems).

V Homology Searching of cDNA Clones and Their Deduced Proteins

The cDNAs of the Sequence Listing or their deduced amino acid sequences were used to query databases such as GenBank, SwissProt, BLOCKS, and the like. These databases that contain previously identified and annotated sequences or domains were searched using BLAST or BLAST2 to produce alignments and to determine which sequences were exact matches or homologs. The alignments were to sequences of prokaryotic (bacterial) or eukaryotic (animal, fungal, or plant) origin. Alternatively, algorithms such as the one described in Smith and Smith (1992, Protein Engineering 5:35-51) could have been used to deal with primary sequence patterns and secondary structure gap penalties. All of the sequences disclosed in this application have lengths of at least 49 nucleotides, and no more than 12% uncalled bases (where N is recorded rather than A, C, G, or T).

As detailed in Karlin and Altschul (1993; Proc Natl Acad Sci 90:5873-5877), BLAST matches between a query sequence and a database sequence were evaluated statistically and only reported when they satisfied the threshold of 10⁻²⁵ for nucleotides and 10⁻¹⁴ for peptides. Homology was also evaluated by product score calculated as follows: the % nucleotide or amino acid identity [between the query and reference sequences] in BLAST is multiplied by the % maximum possible BLAST score [based on the lengths of query and reference sequences] and then divided by 100. In comparison with hybridization procedures used in the laboratory, the stringency for an exact match was set from a lower limit of about 40 (with 1-2% error due to uncalled bases) to a 100% match of about 70.

The BLAST software suite (NCBI, Bethesda Md.; http://www.ncbi.nlm.nih.gov/gorf/bl2.html), includes various sequence analysis programs including “blastn” that is used to align nucleotide sequences and BLAST2 that is used for direct pairwise comparison of either nucleotide or amino acid sequences. BLAST programs are commonly used with gap and other parameters set to default settings, e.g.: Matrix: BLOSUM62; Reward for match: 1; Penalty for mismatch: −2; Open Gap: 5 and Extension Gap: 2 penalties; Gap x drop-off: 50; Expect: 10; Word Size: 11; and Filter: on. Identity is measured over the entire length of a sequence. Brenner et al. (1998; Proc Natl Acad Sci 95:6073-6078, incorporated herein by reference) analyzed BLAST for its ability to identify structural homologs by sequence identity and found 30% identity is a reliable threshold for sequence alignments of at least 150 residues and 40%, for alignments of at least 70 residues.

The cDNAs of this application were compared with assembled consensus sequences or templates found in the LIFESEQ GOLD database (Incyte Genomics). Component sequences from cDNA, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another, and those having local similarity of at least 82% were combined and reassembled. Bins having templates with less than 95% local identity were split. Templates were subjected to analysis by STITCHER/EXON MAPPER algorithms that determine the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of ≦1×10⁻⁸. The templates were also subjected to frameshift FASTx against GENPEPT, and homolog match was defined as having an E-value of ≦1×10⁻⁸. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. No. 08/812,290 and U.S. Ser. No. 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis Mo.; http://pfam.wustl.edu/). The cDNA was further analyzed using MACDNASIS PRO software (Hitachi Software Engineering), and LASERGENE software (DNASTAR) and queried against public databases such as the GenBank rodent, mammalian, vertebrate, prokaryote, and eukaryote databases, SwissProt, BLOCKS, PRINTS, PPAM, and Prosite.

VI Chromosome Mapping

Radiation hybrid and genetic mapping data available from public resources such as the Stanford Human Genome Center (SHGC), Whitehead Institute for Genome Research (WIGR), and Généthon are used to determine if any of the cDNAs presented in the Sequence Listing have been mapped. Any of the fragments of the cDNA encoding ECM-related protein that have been mapped result in the assignment of all related regulatory and coding sequences to the same location. The genetic map locations are described as ranges, or intervals, of human chromosomes. The map position of an interval, in cM (which is roughly equivalent to 1 megabase of human DNA), is measured relative to the terminus of the chromosomal p-arm.

VII Hybridization Technologies and Analyses

Tissue Sample Preparation

Matched normal colon and cancerous colon or colon polyp tissue samples were provided by the Huntsman Cancer Institute, (Salt Lake City, Utah), and are described as follows: Donor 3754, age and sex unknown, pendunculated colon polyp; Donor 3755, age and sex unknowns polyp, family history of colon cancer; Donor 3583, 58 years old, male, tubulovillous adenoma (polyp), patient was also diagnosed with a hyperplastic polyp; Donor 3311, 85 years old, male, invasive, poorly differentiated adenocarcinoma, metastatic, 2/9 lymph nodes positive, TNM classification: T4, N1, Mx, patient was also diagnosed with multiple tubular adenomas; Donor 3839, 60 years old, sex unknown, colon cancer, no pathology report; Donor 4614, 67 years old, sex unknown, colon adenocarcinoma, moderately differentiated, DUKE'S B, TNM classification: T3, NO. In Table 2, Donor samples 3754, 3755, and 3311 were compared against a common control tissue designated Donor 3753, which was a pool of normal colon tissue from 3 donors. All other comparisons were done with matched normal and tumor tissue from the same donor.

Matched normal and lung tumor tissue samples (Donor IDs 5796 and 5800) were obtained from the Roy Castle International Institute for Cancer Research (Liverpool, United Kingdom). Donor 5796 is a 66 year-old male with a squamous cell carcinoma. Donor 5800 is a 75 year-old female with a squamous cell carcinoma.

The following normalized, first-strand, cDNA preparations of human, adult, normal tissues were obtained from Clontech Laboratories Inc. (Palo Alto Calif.): heart, pooled from 16 male/female Caucasians, ages 25-59;; brain (whole), pooled from 4 male Caucasians, ages 43-55; placenta, pooled from 10 female Caucasians, ages 22-35; lung, pooled from 2 female Caucasians, ages 24 and 32; liver, 35-yr-old male Caucasian; skeletal muscle, pooled from 35 male/female Caucasians, ages 20-60; kidney, pooled from 14 male/female Caucasians, ages 24-55; pancreas, pooled from 20 male/female Caucasians, ages 25-59; spleen, pooled from 6 male/female Caucasians, ages 24-61; thymus, pooled from 9 male/female Caucasians, ages 18-32; prostate, pooled from 20 Caucasians, ages 20-58; testis, pooled from 45 Caucasians, ages 14-64; ovary, pooled from 7 Caucasians, ages 17-60; small intestine, pooled from 32 male/female Caucasians, ages 15-57; colon, pooled from 20 male/female Caucasians, ages 17-76 (includes inner mucosal lining); and peripheral blood leukocytes, pooled from male/female Caucasians, ages 18-40, all samples negative for HIV-I, HIV-II, hepatitis B and syphilis.

The HT-29 human colorectal adenocarcinoma cell line was obtained from The American Type Culture Collection (ATCC, Manassas Va.) and was cultured according to the suppliers specifications.

Immobilization of cDNAs on a Substrate

The cDNAs are applied to a substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37C for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning, Acton Mass.) by ultrasound in 0.1% SDS and acetone, etching in 4% hydrofluoric acid (VWR Scientific Products, West Chester Pa.), coating with 0.05% aminopropyl silane (Sigma Aldrich) in 95% ethanol, and curing in a 110C oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford Mass.) for 30 min at 60C; then the arrays are washed in 0.2% SDS and rinsed in distilled water as before.

Probe Preparation for TAQMAN Analysis

Probes for TAQMAN analysis were prepared according to the manfacturer's protocol (Applied Biosystems).

Probe Preparation for Membrane Hybridization

Hybridization probes derived from the cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to IOOC for five min, and briefly centrifuging. The denatured cDNA is then added to a REDIPRIME tube (APB), gently mixed until blue color is evenly distributed, and briefly centrifuged. Five μl of [³²P]dCTP is added to the tube, and the contents are incubated at 37C for 10 min. The labeling reaction is stopped by adding 5 μl of 0.2M EDTA, and probe is purified from unincorporated nucleotides using a PROBEQUANT G-50 microcolumn (APB). The purified probe is heated to 100C for five min, snap cooled for two min on ice, and used in membrane-based hybridizations as described below.

Probe Preparation for Polymer Coated Slide Hybridization

The following method was used for the preparation of probes for the microarray analysis presented in Table 2. Hybridization probes derived from mRNA isolated from samples are employed for screening cDNAs of the Sequence Listing in array-based hybridizations. Probe is prepared using the GEMbright kit (Incyte Genomics) by diluting mRNA to a concentration of 200 ng in 9 μl TE buffer and adding 5 μl 5× buffer, 1 μl 0.1 M DTT, 3 μl Cy3 or Cy5 labeling mix, 1 μl RNase inhibitor, 1 μl reverse transcriptase, and 5 μl 1× yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37C for two hr. The reaction mixture is then incubated for 20 min at 85C, and probes are purified using two successive CHROMA SPIN+TE 30 columns (Clontech, Palo Alto Calif.). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800×g, and the pellet is resuspended in 12 μl resuspension buffer, heated to 65C for five min, and mixed thoroughly. The probe is heated and mixed as before and then stored on ice. Probe is used in high density array-based hybridizations as described below.

Membrane-Based Hybridization

Membranes are pre-hybridized in hybridization solution containing 1% Sarkosyl and 1× high phosphate buffer (0.5 M NaCl, 0.1 M Na₂HPO₄, 5 mM EDTA, pH 7) at 55C for two hr. The probe, diluted in 15 ml fresh hybridization solution, is then added to the membrane. The membrane is hybridized with the probe at 55C for 16 hr. Following hybridization, the membrane is washed for 15 min at 25C in 1 mM Tris (pH 8.0), 1% Sarkosyl, and four times for 15 min each at 25C in 1 mM Tris (pH 8.0). To detect hybridization complexes, XOMAT-AR film (Eastman Kodak, Rochester N.Y.) is exposed to the membrane overnight at −70C, developed, and examined visually.

Polymer Coated Slide-Based Hybridization

The following method was used in the microarray analysis presented in Table 2. Probe is heated to 65C for five min, centrifuged five min at 9400 rpm in a 5415C microcentrifuge (Eppendorf Scientific, Westbury N.Y.), and then 18 μl is aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60C. The arrays are washed for 10 min at 45C in 1×SSC, 0.1% SDS, and three times for 10 min each at 45C in 0.1×SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of genes in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to equal numbers of probes derived from both biological samples give a distinct combined fluorescence (Shalon WO95/35505).

Hybridization complexes are detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light is focused on the array using a 20× microscope objective (Nikon, Melville N.Y.). The slide containing the array is placed on a computer-controled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Filters positioned between the array and the photomultiplier tubes are used to separate the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

Solution-Based Hybridization (TAOMAN)

Hybridization reactions and detection of hybridization complexes for TAQMAN analysis were performed according to the manfacturer's protocol (Applied Biosystems).

VIII Electronic Analysis

BLAST was used to search for identical or related molecules in the GenBank or LIFESEQ databases (Incyte Genomics). The product score for human and rat sequences was calculated as follows: the BLAST score is multiplied by the % nucleotide identity and the product is divided by (5 times the length of the shorter of the two sequences), such that a 100% alignment over the length of the shorter sequence gives a product score of 100. The product score takes into account both the degree of similarity between two sequences and the length of the sequence match. For example, with a product score of 40, the match will be exact within a 1% to 2% error, and with a product score of at least 70, the match will be exact. Similar or related molecules are usually identified by selecting those which show product scores between 8 and 40.

Electronic northern analysis was performed at a product score of 70 and is shown in FIG. 3. All sequences and cDNA libraries in the LIFESEQ database were categorized by system, organ/tissue and cell type. The categories included cardiovascular system, connective tissue, digestive system, embryonic structures, endocrine system, exocrine glands, female and male genitalia, germ cells, hemic/immune system, liver, musculoskeletal system, nervous system, pancreas, respiratory system, sense organs, skin, stomatognathic system, unclassified/mixed, and the urinary tract. For each category, the number of libraries in which the sequence was expressed were counted and shown over the total number of libraries in that category. In a non-normalized library, significant expression may reflect presence or absence or differential expression of the cDNA.

IX Complementary Molecules

Molecules complementary to the cDNA, from about 5 (PNA) to about 5000 bp (complement of a cDNA insert), are used to detect or inhibit gene expression. Detection is described in Example VII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in “triple helix” base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if elements for inducing vector replication are used in the transformation/expression system.

Stable transformation of dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the cDNA encoding the protein.

X Expression of ECM-Related Protein

Expression and purification of the protein are achieved using either a mammalian cell expression system or an insect cell expression system. The pUB6/V5-His vector system (Invitrogen, Carlsbad Calif.) is used to express ECM-related protein in CHO cells. The vector contains the selectable bsd gene, multiple cloning sites, the promoter/enhancer sequence from the human ubiquitin C gene, a C-terminal V5 epitope for antibody detection with anti-V5 antibodies, and a C-terminal polyhistidine (6×His) sequence for rapid purification on PROBOND resin (Invitrogen). Transformed cells are selected on media containing blasticidin.

Spodoptera frugiperda (Sf9) insect cells are infected with recombinant Autographica californica nuclear polyhedrosis virus (baculovirus). The polyhedrin gene is replaced with the cDNA by homologous recombination and the polyhedrin promoter drives cDNA transcription. The protein is synthesized as a fusion protein with 6×his which enables purification as described above. Purified protein is used in the following activity and to make antibodies

XI Production of Antibodies

ECM-related protein is purified using polyacrylamide gel electrophoresis and used to immunize mice or rabbits. Antibodies are produced using the protocols below. Alternatively, the amino acid sequence of ECM-related protein is analyzed using LASERGENE software (DNASTAR) to determine regions of high antigenicity. An antigenic epitope, usually found near the C-terminus or in a hydrophilic region is selected, synthesized, and used to raise antibodies. Typically, epitopes of about 15 residues in length are produced using an ABI 431A peptide synthesizer (Applied Biosystems) using Fmoc-chemistry and coupled to KLH (Sigma-Aldrich) by reaction with N-maleimidobenzoyl-N-hydroxysuccinimide ester to increase antigenicity.

Rabbits are immunized with the epitope-KLH complex in complete Freund's adjuvant. Immunizations are repeated at intervals thereafter in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washing, and reacting with radio-iodinated goat anti-rabbit IgG. Methods well known in the art are used to determine antibody titer and the amount of complex formation.

XII Purification of Naturally Occurring Protein Using Specific Antibodies

Naturally occurring or recombinant protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIII Screening Molecules for Specific Binding with the cDNA or Protein

The cDNA, or fragments thereof, or the protein, or portions thereof, are labeled with ³²P-dCTP, Cy3-dCTP, or Cy5-dCTP (APB), or with BIODIPY or FITC (Molecular Probes, Eugene Oreg.), respectively. Libraries of candidate molecules or compounds previously arranged on a substrate are incubated in the presence of labeled cDNA or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XIV Two-Hybrid Screen

A yeast two-hybrid system, MATCHMAKER LexA Two-Hybrid system (Clontech Laboratories, Palo Alto Calif.), is used to screen for peptides that bind the protein of the invention. A cDNA encoding the protein is inserted into the multiple cloning site of a pLexA vector, ligated, and transformed into E. coli. cDNA, prepared from MRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] cells using a polyethylene glycol/lithium acetate protocol. Transformed yeast cells are plated on synthetic dropout (SD) media lacking histidine (-His), tryptophan (-Trp), and uracil (-Ura), and incubated at 30C until the colonies have grown up and are counted. The colonies are pooled in a minimal volume of 1×TE (pH 7.5), replated on SD/-His/-Leu/-Trp/-Ura media supplemented with 2% galactose (Gal), 1% raffinose (Raf), and 80 mg/ml 5-bromo-4-chloro-3-indolyl β-d-galactopyranoside (X-Gal), and subsequently examined for growth of blue colonies. Interaction between expressed protein and cDNA fusion proteins activates expression of a LEU2 reporter gene in EGY48 and produces colony growth on media lacking leucine (-Leu). Interaction also activates expression of β-galactosidase from the p8op-lacZ reporter construct that produces blue color in colonies grown on X-Gal.

Positive interactions between expressed protein and cDNA fusion proteins are verified by isolating individual positive colonies and growing them in SD/-Trp/-Ura liquid medium for 1 to 2 days at 30C. A sample of the culture is plated on SD/-Trp/-Ura media and incubated at 30C until colonies appear. The sample is replica-plated on SD/-Trp/-Ura and SD/-His/-Trp/-Ura plates. Colonies that grow on SD containing histidine but not on media lacking histidine have lost the pLexA plasmid. Histidine-requiring colonies are grown on SD/Gal/Raf/X-Gal/-Trp/-Ura, and white colonies are isolated and propagated. The pB42AD-cDNA plasmid, which contains a cDNA encoding a protein that physically interacts with the protein, is isolated from the yeast cells and characterized.

XV ECM-Related Protein Assay

ECMRP binding activity is determined in a ligand-binding assay using candidate ligand molecules in the presence of ¹²⁵I-labeled ECM-related protein. ECM-related protein is labeled with ¹²⁵I Bolton-Hunter reagent (Bolton and Hunter (1973) Biochem J 133:529-539). Candidate ligand molecules, previously arrayed in the wells of a multi-well plate, are incubated with the labeled ECM-related protein, washed, and any wells with labeled ECM-related protein complex are assayed. Data obtained using different concentrations of ECM-related protein are used to calculate values for the number, affinity, and association of ECM-related protein with candidate ligand molecules.

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

TABLE 1

| Tissue | #cDNAs | Found | Abund | % Abund |
|---|---|---|---|---|
| Cardiovascular System | 270162 | 1/72 | 1 | 0.0004 |
| Connective Tissue | 147886 | 1/54 | 1 | 0.0007 |
| Digestive System | 514430 | 6/151 | 7 | 0.0014 |
| Embryonic Structures | 107325 | 2/23 | 2 | 0.0019 |
| Endocrine System | 233587 | 3/63 | 4 | 0.0017 |
| Exocrine Glands | 255105 | 4/64 | 5 | 0.0020 |
| Genitalia, Female | 445078 | 3/113 | 3 | 0.0007 |
| Genitalia, Male | 453150 | 5/118 | 7 | 0.0015 |
| Germ Cells | 46185 | 0/5 | 0 | 0.0000 |
| Hemic and Immune System | 701709 | 1/166 | 1 | 0.0001 |
| Liver | 110945 | 1/34 | 1 | 0.0009 |
| Musculoskeletal System | 162794 | 0/50 | 0 | 0.0000 |
| Nervous System | 973795 | 0/221 | 0 | 0.0000 |
| Pancreas | 111757 | 2/25 | 3 | 0.0027 |
| Respiratory System | 407942 | 7/95 | 9 | 0.0022 |
| Sense Organs | 25346 | 0/10 | 0 | 0.0000 |
| Skin | 72110 | 1/15 | 4 | 0.0055 |
| Stomatognathic System | 14025 | 1/11 | 1 | 0.0071 |
| Unclassified/Mixed | 150146 | 2/19 | 2 | 0.0013 |
| Urinary Tract | 287931 | 2/66 | 2 | 0.0007 |
| Totals | 5321883 | 33/1292 | 55 | 0.0000 |

TABLE 2

| DE | P1Description | P2Description |
|---|---|---|
| 1.7 | Human, Colon, Nrml, mw/ | Human, Colon Tumor, |
| | AdenoCA, Dn4614 | AdenoCA, Dn4614 |
| 2.3 | Human, Colon Pool, Nrml, | Human, Colon, Polyp, |
| | Dn3753 | Dn3755 |
| 4.4 | Human, Colon Pool, Nrml, | Human, Colon Tumor, |
| | Dn3753 | Cancer, Dn3311 |
| 1.7 | Human, Colon Pool, Nrml, | Human, Colon, Polyp, |
| | Dn3753 | Dn3754 |
| 2.5 | Human, Colon Pool, Nrml, mw/ | Human, Colon Tumor, |
| | Adenoma, Dn3583 | Adenoma, Dn3583 |
| 3.3 | Human, Colon, Nrml, mw/ | Human, Colon Tumor, |
| | AdenoCA, Dn3839 | AdenoCA Dn3839 |
| 3.8 | Human, Colon, Rectum, Nrml, mw/ | Human, Colon Tumor, |
| | Cancer, Dn3581 | Rectum, Cancer, Dn3581 |
| 1.5 | Human, Lung, Nrml, mw/ | Human, Lung Tumor, |
| | Squamous Cell CA, Dn5796 | Squamous Cell CA, Dn5796 |
| 3.4 | Human, Lung, Nrml, mw/ | Human, Lung Tumor, |
| | Squamous Cell CA, Dn5800 | Squamous Cell CA, Dn5800 |

What is claimed is:

1. An isolated cDNA comprising a nucleic acid sequence encoding a protein having the amino acid sequence of SEQ ID NO: 1, or the complement of the cDNA.

2. An isolated cDNA comprising a nucleic acid sequence selected from:
a) SEQ ID NO:2 or the complement thereof;
b) a fragment of SEQ ID NO:2 selected from SEQ ID NOs:3-13 or the complement thereof; and
c) a variant of SEQ ID NO:2 selected from SEQ ID NOs:14-15 or the complement thereof.

3. A composition comprising the cDNA of claim 1 and a labeling moiety.

4. A vector comprising the cDNA of claim 1.

5. A host cell comprising the vector of claim 4.

6. A method for using a cDNA to produce a protein, the method comprising:
a) culturing the host cell of claim 5 under conditions for protein expression; and
b) recovering the protein from the host cell culture.

7. A method for using a cDNA to detect expression of a nucleic acid in a sample comprising:
a) hybridizing the composition of claim 3 to nucleic acids of the sample under conditions to form at least one hybridization complex; and
b) detecting hybridization complex formation, wherein complex formation indicates expression of the cDNA in the sample.

8. The method of claim 7 further comprising amplifying the nucleic acids of the sample prior to hybridization.

9. The method of claim 7 wherein the composition is attached to a substrate.

10. The method of claim 7 wherein complex formation is compared with at least one standard to determine differential expression.

11. A method of using a cDNA to screen a plurality of molecules or compounds, the method comprising:
a) combining the cDNA of claim 1 with a plurality of molecules or compounds under conditions to allow specific binding; and
b) detecting specific binding, thereby identifying a molecule or compound which specifically binds the cDNA.

12. The method of claim 11 wherein the molecules or compounds are selected from DNA molecules, RNA molecules, peptide nucleic acids, artificial chromosome constructions, peptides, transcription factors, repressors, and regulatory molecules.

13. A purified protein or a portion thereof produced by the method of claim 6 and selected from:
a) an amino acid sequence of SEQ ID NO:1;
b) an antigenic epitope of SEQ ID NO: 1; and
c) a biologically active portion of SEQ ID NO:1.

14. A composition comprising the protein of claim 13 and a pharmaceutical carrier.

15. A method for using a protein to screen a plurality of molecules or compounds to identify at least one ligand, the method comprising:
a) combining the protein of claim 13 with the molecules or compounds under conditions to allow specific binding; and
b) detecting specific binding, thereby identifying a ligand which specifically binds the protein.

16. The method of claim 15 wherein the molecules or compounds are selected from DNA molecules, RNA molecules, peptide nucleic acids, peptides, proteins, mimetics, agonists, antagonists, antibodies, immunoglobulins, inhibitors, and drugs.

17. A method of using a protein to prepare and purify antibodies comprising:
a) immunizing an animal with the protein of claim 15 under conditions to elicit an antibody response;
b) isolating animal antibodies;
c) attaching the protein to a substrate;
d) contacting the substrate with isolated antibodies under conditions to allow specific binding to the protein;
e) dissociating the antibodies from the protein, thereby obtaining purified antibodies.

18. An antibody produced by the method of claim 17.

19. A method for using an antibody to diagnose conditions or diseases associated with expression of a protein, the method comprising:
a) combining the antibody of claim 18 with a sample, thereby forming antibody:protein complexes; and
b) comparing complex formation with a standard, wherein the comparison indicates expression of the protein in the sample.

20. The method of claim 19 wherein expression is diagnostic of a colon or lung cancer.

21. A pharmaceutical composition comprising the antibody of claim 18 and a pharmaceutical carrier.

22. A method for treating a cancer comprising administering to a person in need of such treatment an effective amount of the composition of claim 21.

23. The method of claim 22 wherein the cancer is a colon cancer.

24. The method of claim 22 wherein the cancer is a lung cancer.


SEQUENCE LISTING (15 sequences)

SEQ ID NO: 1 - protein, 546 aa, Homo sapiens [sequence omitted]

SEQ ID NO: 2 - DNA, 3224 bp, Homo sapiens
  misc_feature: Incyte ID No 2743093CB1
gatgctattgagagtcttcctccaaacatcttccgatttgttcctttaacccatctagatcttcgtggaaatcaattacaaacattgccttatgttggttttctcgaacacattggccgaatattggatcttcagttggaggacaacaaatgggcctgcaattgtgacttattgcagttaaaaacttggttggagaacatgcctccacagtctataacttggtgatgttgtctgcaacagccctccattttttaaaggaagtatactcagtagactaaagaaggaatctatttgccctactccaccagtgtatgaagaacatgaggatccttcaggatcattacatctggcagcaacatcttcaataaatgatagtcgcatgtcaactaagaccacgtccattctaaaactacccaccaaagcaccaggtttgataccttatattacaaagccatccactcaacttccaggaccttactgccctattccttgtaactgcaaagtcctatccccatcaggacttctaatacattgtcaggagcgcaacattgaaagcttatcagatctgagacctcctccgcaaaatcctagaaagctcattctagcgggaaatattattcacagtttaatgaagtctgatctagtggaatatttcactttggaaatgcttcacttgggaaacaatcgtattgaagttcttgaagaaggatcgtttatgaacctaacgagattacaaaaactctatctaaatggtaaccacctgaccaaattaagtaaaggcatgttccttggtctccataatcttgaatacttatatcttgaatacaatgccattaaggaaatactgccaggaacctttaatccaatgcctaaacttaaagtcctgtatttaaataacaacctcctccaagttttaccaccacatattttttcaggggttcctctaactaaggtaaatcttaaaacaaaccagtttacccatctacctgtaagtaatattttggatgatcttgatttactaacccagattgaccttgaggataacccctgggactgctcctgtgacctggttggactgcagcaatggatacaaaagttaagcaagaacacagtgacagatgacatcctctgcacttcccccgggcatctcgacaaaaaggaattgaaagccctaaatagtgaaattctctgtccaggtttagtaaataacccatccatgccaacacagactagttaccttatggtcaccactcctgcaacaacaacaaatacggctgatactattttacgatctcttacggacgctgtgccactgtctgttctaatattgggacttctgattatgttcatcactattgttttctgtgctgcagggatagtggttcttgttcttcaccgcaggagaagatacaaaaagaaacaagtagatgagcaaatgagagacaacagtcctgtgcatcttcagtacagcatgtatggccataaaaccactcatcacactactgaaagaccctctgcctcactctatgaacagcacatggtgagccccatggttcatgtctatagaagtccatcctttggtccaaagcatctggaagaggaagaagagaggaatgagaaagaaggaagtgatgcaaaacatctccaaagaagtcttttggaacaggaaaatcattcaccactcacagggtcaaatatgaaatacaaaaccacgaaccaatcaacagaatttttatccttccaagatgccagctcattgtacagaaacattttagaaaaagaaagggaacttcagcaactgggaatcacagaatacctaaggaaaaacattgctcagctccagcctgatatggaggcacattatcctggagcccacgaagagctgaagttaatggaaacattaatgtactcacgtccaaggaaggtattagtggaacagacaaaaaatgagtattttgaacttaaagctaatttacatgctgaacctgactatttagaagtcctggagcagcaaacatagatggagagtttgagggctttcgcagaaatgctgtgattctgttttaagtccataccttgtaaataagtgccttacgtgagtgtgtcatcaatcagaacctaagcacagcagtaaactatggggaaaaaaaaagaagaagaaaaagaaactcagggatcactgggagaagccatggcattatcttcaggcaatttagtctgtcccaaataaaataaatccttgcatgtaaatcattcaagggttatagtaatatttcatatactgaaaagtgtctcataggagtcctcttgcacatctaaaaaggctgaacatttaagtatcccgaattttcttgaattgctttccctatagattaattacaattggatttcatcatttaaaaaccatacttgtatatgtagttataatatgtaaggaatacattgtttataaccagtatgtacttcaaaaatgtgtattgtcaaacatacctaactttcttgcaataaatgcaaaagaaactggaacttgacaattataaatagtaatagtgaagaaaaaatagaaaggttgcaattatataggccatgggtggctcaaaactttgaacatttgagcttaaacaaatgccactctcatgcattctaaattaaaaagttaaaatgattaatagttcaggtggaagaaataagcatactttttgggttttctacacattttgtgtagacaattttaatgtcagtgctgctgtgaactaaagtatgtcatttatgctcaaagtttaattcttcttcttgggatattttaaaaatgctactgagattctgctgtaaatatgactagagaatatattgggtttgctttatttcataggcttaattctttgtaaatctgaatgaccataatagaaatacatttcttgtggcaagtaattcacagttgtaaagtaaataggaaaaattattttatttttattgatgtacattgatagatgccataaatcagtagcaaaaggcacttctaaaggtaagtggtttaagttgcctcaagagagggacaatgtagctttattttacaagaaggcatagttagatttctatgaaatatttattctgtacagttttatatatttttggttcacaaaagtaattattcttgggtgcctttcaagaaaattaaaaatactacccactacaataaaactaaaatgaaaactcaaaaaaaa

SEQ ID NO: 3 - DNA, 246 bp, Homo sapiens
  misc_feature: Incyte ID No 2743093H1
tgcttcacttgggaaacaatcgtattgaagttcttgaagaaggatcgtttatgaacctaacgagattacaaaaactctatctaaatggtaaccacctgaccaaattaagtaaaggcatgttccttggtctccataatcttgaatacttatatcttgaatacaatgccattaaggaaatactgccaggaacctttaatccaatgcctaaacttaaagtcctgtatttaaataacaacctcctccaag

SEQ ID NO: 4 - DNA, 597 bp, Homo sapiens
  misc_feature: Incyte ID No 4876623F9
cctattccttgtaactgcaaagtcctatccccatcaggacttctaatacattgtcaggagcgcaacattgaaagcttatcagatctgagacctcctccgcaaaatcctagaaagctcattctagcgggaaatattattcacagtttaatgaagtctgatctagtggaatatttcactttggaaatgcttcacttgggaaacaatcgtattgaagttcttgaagaaggatcgtttatgaacctaacgagattacaaaaactctatctaaatggtaaccacctgaccaaattaagtaaaggcatgttccttggtctccataatcttgaatacttatatcttgaatacaatgccattaaggaaatactgccaggaacctttaatccaatgcctaaacttaaagtcctgtatttaaataacaacctcctccaagtttaaccaccacatattttttcaggggttcctcataactaaggtaaatcttaaaacaaaccagtttacccatctacctgtaagtaatatttctggatgatcttgatttactaacccagattgaccttgaggataacccctgggactgctcctgtgacctggttggac

SEQ ID NO: 5 - DNA, 510 bp, Homo sapiens
  misc_feature: Incyte ID No 2316239T6
tccccatagtttactgctgtgcttaggttctgattgatgacacactcacgtaaggcacttatttacaaggtatggacttaaaacagaatcacagcatttctgcgaaagccctcaaactctccatctatgtttgctgctccaggacttctaaatagtcaggttcagcatgtaaattagctttaagttcaaaatactcattttttgtctgttccactaataccttccttggacgtgagtacattaatgtttccattaacttcagctcttcgtgggctccaggataatgtgcctccatatcaggctggagctgagcaatgtttttccttaggtattctgtgattcccagttgctgaagttccctttctttttctaaaatgtttctgtacaatgagctggcatcttggaaggataaaaattctgttgattggttcgtggttttgtatttcatatttgaccctgtgagtggtgaatgattttcctgttncaaaagacttctttggagatgttttg

SEQ ID NO: 6 - DNA, 549 bp, Homo sapiens
  misc_feature: Incyte ID No 6258015F8
aaaacttggaggaggttgttatttaaatacaggactttaagtttaggcattggattaaaggttcctggcagtatttccttaatggcattgtattcaagatataagtattcaagattatggagaccaaggaacatgcctttacttaatttggtcaggtggttaccatttagatagagtttttgtaatctcgattaggttcataaacgatccttcttcaagaacttcaatacgattgtttcccaagtgaagcatttccaaagtgaaatattccactagatcagacttcattaaactgtgaataatatttcccgctagaatgagctttctaggattttgcggaggaggtctcagatctgataagctttcaatgttgcgctcctgacaatgtattagaagtcctgatggggataggactttgcagttacaaggaatagggcagtaaggtcctggaagttgagtggatggctttgtaatataaggtatcaaacctggtgctttggtgggtagttttagaatggacgtggtcttagttgacatgcgactatcatt

SEQ ID NO: 7 - DNA, 466 bp, Homo sapiens
  misc_feature: Incyte ID No 7677606J1
ggaagttgagtggatggcttagtaatataaggtatcaaacctggtgctttggtgggtagttttagaatggacgtggtcttagttgacatgcgactatcatttattgaagatgttgctgccagatgtaatgatcctgaaggatcctcatgttcttcatacactggtggagtagggcaaatagattccttctttagtctactgagtatacttcctttaaaaaatggagggctgttgcagacaacatcaccaagttatagactgtggaggcatgttctccaaccaagtttttaactgcaataagtcacaattgcaggcccatttgttgtcctccaactgaagatccaatattcggccaatgtgttcgagaaaaccaacataaggcaatgtttgtaattgatttccacgaagatctagatgggttaaaggaacaaatcggaagatgtttggaggaagactctcaatagca

SEQ ID NO: 8 - DNA, 640 bp, Homo sapiens
  misc_feature: Incyte ID No 71111915V1
aatcttgaatacttatatcttgaatacaatgccattaaggaaatactgccaggaacctttaatccaatgcctaaacttaaagtcctgtatttaaataacaacctcctccaagttttaccaccacatattttttcaggcgttcctctaactaaggtaaatcttaaaacaaaccagtttacccatctacctgtaagtaatattttggatgatcttgatttactaacccagattgaccttgaggataacccctgggactgctcctgtgacctggttggactgcagcaatggatacaaaagttaagcaagaacacagtgacagatgacatcctctgcacttcccccgggcatctcgacaaaaaggaattgaaagccctaaatagtgaaattctctgtccaggtttagtaaataacccatccatgccaacacagactagttaccttatggtcaccactcctgcaacaacaacaaatacggctgatactattttacgatctcttacggacgctgtgccactgtctgttctaatattgggacttctgattatgttcatcactattgttttctgtgctgcagggatagtggttcttgttcttcaccgcaggagaagatacaaaagaaacaagtagatgagcaatgaga

SEQ ID NO: 9 - DNA, 638 bp, Homo sapiens
  misc_feature: Incyte ID No 71112850V1
caatatattctctagtcatatttacagcagaatctcagtagcatttttaaaatatcccaagaagaagaattaaactttgagcataaatgacatactttagtttcacagcagcactgacattaaaattgtctacacaaaatgtgtagaaaacccaaaaagtatgcttatttcttccacctgaactattaatcattttaactttttaatttagaatgcatgagagtggcatttgtttaagctcaaatgttcaaagttttgagccacccatggcctatataattgcaacctttctattttttcttcactattactatttataattgtcaagttccagtttcttttgcatttattgcaagaaagttaggtatgtttgacaatacacatttttgaagtacatactggttataaacaatgtattccttacatattataactacatatacaagtatggtttttaaatgatgaaatccaattgtaattaatctatagggaaagcaattcaagaaaattcgggatacttaaatgttcagcctttttagatgtgcaagaggactcctatgagacacttttcagtatatgaaatattactataaccttgaatgatttacatgcgagatctattttattgggacagacta

SEQ ID NO: 10 - DNA, 582 bp, Homo sapiens
  misc_feature: Incyte ID No 71262960V1
gcagcaatggtatacaaaagttaagcaagaacacagtgacagatgacatcctctgcacttcccccgggcatctcgacaaaaaggaattgaaagccctaaatagtgaaattctctgtccaggtttagtaaataacccatccatgccaacacagactagttaccttatggtcaccactcctgcaacaacaacaaatacggctgatactattttacgatctcttacggacgctgtgccactgtctgttctaatattgggacttctgattatgttcatcactattgttttctgtgctgcagggatagtggttcttgttcttcaccgcaggagaagatacaaaaagaaacaagtagatgagcaaatgagagacaacagtcctgtgcatcttcagtacagcatgtatggccataaaaccactcatcacactactgaaagaccctctgcctcactctatgaacagcacatggtgagccccatggttcatgtctatagaagtccatcctttggtccaaagcatctggaagaggaagaagagaggaatgagaaagaaggaagtgatgcaaaacatctccaaagaagtcttt

SEQ ID NO: 11 - DNA, 494 bp, Homo sapiens
  misc_feature: Incyte ID No 71264035V1
ttttgtgtagacaattttaatgtcagtgctgctgtgaactaaagtatgtcatttatgctcaaagtttaattcttcttcttgggatattttaaaaatgctactgagattctgctgtaaatatgactagagaatatattgggtttgctttatttcataggcttaattctttgtaaatctgaatgaccataatagaaatacatttcttgtggcaagtaattcacagttgtaaagtaaataggaaaaattattttatttttattgatgtacattgatagatgccataaatcagtagcaaaaggcacttctaaaggtaagtggtttaagttgcctcaagagagggacaatgtagctttattttacaagaaggcatagttagatttctatgaaatatttattctgtacagttttatatatttttggttcacaaaagtaattattcttgggtgcctttcaagaaaattaaaaatactacccactacaataaaactaaaatg

SEQ ID NO: 12 - DNA, 444 bp, Homo sapiens
  misc_feature: Incyte ID No 71113484V1
taagcctatgaaataaagcaaacccaatatattctctagtcatatttacagcagaatctcagtagcatttttaaaatatcccaagaagaagaattaaactttgagcataaatgacatactttagtttcacagcagcactgacattaaaatgtctacacaaaatgtgtagaaaacccaaaaagtatgcttatttcttccacctgaactattaatcattttaactttttaatttagaatgcatgagagtggcatttgtttaagctcaaatgttcaaagttttgagccacccatggcctatataattgcaacctttctattttttcttcactattactatttataattgtcaagttccagtttcttttgcatttattgcaagaaagttaggtatgtttgacaatacacatttttgaagtacatactggttataaacaatgtattcct

SEQ ID NO: 13 - DNA, 657 bp, Homo sapiens
  misc_feature: Incyte ID No 71114738V1
caggctcaatatgaaatacaaaaccacgaaccaatcaacagaatttttatccttccaagatgccagctcattgtacagaaacattttagaaaaagaaagggaacttcagcaactgggaatcacagaatacctaaggaaaaacattgctcagctccagcctgatatggaggcacattatcctggagcccacgaagagctgaagttaatggaaacattaatgtactcacgtccaaggaaggtattagtggaacagacaaaaaatgagtattttgaacttaaagctaatttacatgctgaacctgactatttagaagtcctggagcagcaaacatagatggagagtttgagggctttcgcagaaatgctgtgattctgttttaagtccataccttgtaaataagtgccttacgtgagtgtgtcatcaatcagaacctaagcacagcagtaaactatggggaacaaaaaaagaagaagaaaagaaactcagggatcactgggagaagccatggcattatcttcaggcaatttagtctgtcccaaataaaataaatccttgcatgtaaatcattcagggttatagtaatatttcatatactgaaaagtgtctcatagggagtcctcttgcacatctaaaaaggctgacatttaagtatccga

SEQ ID NO: 14 - DNA, 610 bp, Canis familiaris
  misc_feature: Incyte ID No 703545995J1
caaccaaatttttagctctaataagtcacaattacaggtccacttatgtcctccaactggagatccaatatccggccaatgtgttctaagaaaccaacgtaaggcaatgttgcaactgatttccacgaagatctagatgggttaaaggaacaaatcgaaatatgtttggaggaagactctcaatagcattgtcatttaaaattaacactttaagtctgttgagcttgctaaaggcacttggttcaatcactgtaatgaagttgttgtctgcttgtaggaattctaggttttccagtccatggaaggtatcctctttaagaatttctaaagaattgtgattgatgtgaagttgcttaagaaggccaaggccattaaatgcaccagtctcaatatctgcaatgttgttaaatccaagggtgtaatgagatggcattagtaagcccagaaaagtcatttgtgtgaagcattgtcaaaccattatttaataaacttaggtggaaaggtcgtgatggcggcacacttatttgggataacttcttgatacctttctcttcacagtttattagcattgtgccatcttttttcctcacaattgcaaagagaatcacag

SEQ ID NO: 15 - DNA, 273 bp, Mus musculus
  misc_feature: Incyte ID No 112357_Mm.1
tgtgaagtgcattgttcaacttatttttttttcattgacataccctgcgtaggtcgtctagcttagtagcaagaggcgtttctacaggtaagtgttttgagcttcctcaggagagggacagtgtagctttattttacaagaaggtatcactagatttttatgaattatttattttgtacagttttgtatatttttggttcacaagataattatacttgggtgccttttaagaaagtttaagattattgctcacttcaataaaagtaaaatgaa